(19) United States
(12) Patent Application Publication
(10) Pub. No.: US 2024/0142454 A1
(43) Pub. Date: May 2, 2024

(54) ANTIBODIES FOR BINDING PATHOLOGIC FORMS OF CALCINEURIN

(71) Applicant: University of Kentucky Research Foundation, Lexington, KY (US)

(72) Inventors: Susan D. Kraner, Lexington, KY (US); Rodney P. Guttmann, Contonment, FL (US); Christopher M. Norris, Lexington, KY (US); Jenna Leigh Gollihue, Lexington, KY (US)

(21) Appl. No.: 18/520,159

(22) Filed: Nov. 27, 2023

Related U.S. Application Data
(63) Continuation-in-part of application No. 17/325,085, filed on May 19, 2021, now Pat. No. 11,827,718.
(60) Provisional application No. 63/028,079, filed on May 21, 2020.

(51) Int. Cl. G01N 33/573; C07K 16/40; G01N 33/50

(52) U.S. Cl. CPC G01N 33/573; C07K 16/40; G01N 33/5091

(57) ABSTRACT

Provided herein are antibodies, or antigen-binding portions thereof, which specifically bind to pathologic forms of calcineurin. The invention further provides a method of obtaining such antibodies and nucleic acids encoding the same. The invention further relates to compositions and methods for use of these antibodies, including methods for staining pathological tissue.

### RELATED APPLICATIONS

This application is a continuation-in-part of U.S. Pat. No. 11,827,718 issuing from U.S. patent application Ser. No. 17/325,085 filed May 19, 2021, which claims priority from U.S. Provisional Application Ser. No. 63/028,079 filed May 21, 2020, the entire disclosure of which is incorporated herein by this reference.

### GOVERNMENT INTEREST

This invention was made with government support under grant number RF1-AG027297, PO1AG078116, P30AG072946, and AG074146 awarded by the National Institutes of Health. The government has certain rights in the invention.

### REFERENCE TO AN ELECTRONIC SEQUENCE LISTING

The contents of the electronic sequence listing (Kraner UKRF 2425 US2 Sequence Listing.xml; Size: 18,459 bytes; and Date of Creation: Nov. 22, 2023) is herein incorporated by reference in its entirety.

### TECHNICAL FIELD

The presently-disclosed subject matter relates to antibodies that are useful for binding pathologic forms of calcineurin.

### INTRODUCTION

Calcineurin (CN) is a Ca²⁺/calmodulin-dependent protein phosphatase ubiquitously expressed in most mammalian tissues, but found at especially high levels in brain (Aramburu, Rao et al. 2000, Nguyen and Di Giovanni 2008, Vihma, Pruunsild et al. 2008, Baumgartel and Mansuy 2012) where it links increases in intracellular Ca²⁺ to a broad range of processes. In neurons, CN is a key regulator of synaptic plasticity through dephosphorylation of numerous substrates associated with the neuronal cytoskeleton, neurotransmitter receptor trafficking, and Ca²⁺ channels (Malinow, Madison et al. 1988, Lisman 1989, Mulkey, Herron et al. 1993, Wang and Kelly 1996, Foster and Norris 1997). In glial cells, CN regulates immune inflammatory signaling and glutamate regulation through interactions with the nuclear factor of activated T cells (NFAT), and other transcription factors (Norris, Kadish et al. 2005, Fernandez, Fernandez et al. 2007, Canellada, Ramirez et al. 2008, Perez-Ortiz, Serrano-Perez et al. 2008, Sama, Mathis et al. 2008, Nagamoto-Combs and Combs 2010, Shiratori, Tozaki-Saitoh et al. 2010, Furman and Norris 2014, Sompol, Furman et al. 2017).

Ca²⁺ dysregulation, common to brain aging, Alzheimer's disease (AD) and other AD-related dementias (Gibson and Peterson 1987, Landfield 1987, Khachaturian 1989, Disterhoft, Moyer et al. 1994, Foster and Norris, 1997, Norris, Blalock et al. 2006, Thibault, Gant et al. 2007) is thought to disrupt cellular signaling and increase vulnerability to disease through many pathways, including those regulated by CN (Reese and Taglialatela 2011, Sompol, Norris 2018). Indeed, many studies have shown that expression and/or activity levels of CN are elevated in human brain tissue at early and late stages of AD (e.g. Liu, Grundke-Iqbal et al. 2005, Abdul, Sama et al. 2009, Wu, Hudry et al. 2010, Mohmmad Abdul, Baig et al. 2011, Pleiss, Sompol et al. 2016), or in experimental models of AD and ADRDs (e.g. Norris, Kadish et al. 2005, Dineley, Hogan et al. 2007, Reese, Zhang et al. 2008, Dineley, Kayed et al. 2010, Sompol, Furman et al. 2017, Sompol, Gollihue et al. 2023). Furthermore, blocking CN signaling through pharmacologic or genetic approaches ameliorates numerous pathophysiologic and cognitive phenotypes related to AD (Reese and Taglialatela 2011, Sompol and Norris 2018, Kraner and Norris 2018).

In addition to regulation by Ca²⁺/calmodulin, CN may undergo aberrant Ca²⁺-dependent proteolytic activation in diseased and/or injurious states (Norris, 2014, Schultz B, Taday et al. 2021). Proteolysis is mediated by calpains and occurs primarily near the carboxyl terminus of the CN A catalytic subunit (Wu, Tomizawa et al. 2004) leading to the generation of several CN fragments (ΔCNs). Unlike full-length CN (˜60 kDa), ΔCNs exhibit constitutive activity, even in the absence of Ca²⁺ due to the removal or disruption of a critical C terminus autoinhibitory domain (AID). CN fragments, with molecular masses of 45, 48, and 57 kDa, have been observed in human AD tissue (Liu F, Grundke-Iqbal et al. 2005, Wu, Hudry et al. 2010, Pleiss, Sompol et al. 2016) and may appear very early in the disease process (Mohmmad Abdul, Baig et al. 2011).

In neural cells, overexpression of similar ΔCNs can recapitulate AD-like phenotypes including Ca²⁺ dysregulation (Norris, Blalock et al. 2010), synapse dysfunction or degeneration (Winder, Mansuy et al. 1998, Wu, Hudry et al. 2010, Pleiss, Sompol et al. 2016), upregulation of inflammatory cytokines and/or inflammatory transcriptional signatures (Norris, Kadish et al. 2005, Fernandez, Fernandez et al. 2007, Sama, Mathis et al. 2008) and cognitive decline (Mansuy, Winder et al. 1998).

The epitope identified by most commercially-available CN antibodies is located in the carboxy termus region of CN. These C-terminus antibodies detect full length (normal) CN, but do not detect delta-CN. There have been a few attempts to provide an antibody that recognizes an epitope in the amino terminus of CN. These N-terminus antibodies are non-specific, in that they bind both full length CN and delta-CN.

Use of N terminus antibodies in Western blot applications has revealed high levels of delta-CN in postmortem human brain homogenates at early stages of Alzheimer's disease. Similar elevations in delta-CN are found in rodent models of Alzheimer's, stroke, traumatic brain injury, and other forms of neurodegeneration. N-terminus antibodies are useful for detecting pathological CN in Western blots, but cannot distinguish between full length CN and pathological delta-CN in immunohistochemistry and ELISA applications.

Such limitations associated with currently-available CN antibodies have made it impossible to determine where pathologic delta-CN fragments are generated. Thus, it remains unknown whether delta-CN is generated in specific cell types or in close proximity to other forms of neuropathology. Furthermore, currently-available CN antibodies cannot be used to effectively determine how delta-CN mechanistically contributes to brain pathophysiology.

Accordingly, there remains a need in the art for an antibody that does not bind full-length/normal CN, but is specific for pathologic delta-CN fragments, which can be used in conjunction with immunohistochemistry to determine where delta-CN is generated in relation to different cell types and/or to study developing neuropathology.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes monoclonal antibodies that bind pathologic delta-CN fragments, but do not bind full-length (normal) CN. In conjunction with immunohistochemistry, antibodies of the instant invention can pinpoint where delta-CN is generated in relation to different cell types and/or developing neuropathology.

The presently-disclosed subject matter provides antibodies, or antigen-binding portions thereof, which specifically bind to pathologic forms of calcineurin. The invention further provides a method of obtaining such antibodies and nucleic acids encoding the same. The invention further relates to compositions and methods for use of these antibodies.

The presently-disclosed subject matter includes isolated nucleic acid molecules. In some embodiments, the isolated nucleic acid molecule encoding a heavy chain, a light chain, or both a heavy chain and a light chain of an antibody or an antigen-binding portion thereof that specifically binds pathologic forms of calcineurin. In some embodiments, the isolated nucleic acid molecule comprises: (a) the nucleotide sequence of SEQ ID NO: 2; (b) the nucleotide sequence of SEQ ID NO: 4; (c) the nucleotide sequence pairs SEQ ID NOs: 2 and 4; (d) the nucleotide sequence of SEQ ID NO: 6; (e) the nucleotide sequence of SEQ ID NO: 8; or (f) the nucleotide sequence pairs SEQ ID NOs: 6 and 8.

The presently-disclosed subject matter includes vectors. In some embodiments, the vector comprises a nucleic acid as disclosed herein. The presently-disclosed subject matter includes host cells. In some embodiments, the host cell comprises a nucleic acid as disclosed herein or a vector as disclosed herein. In some embodiments, the host cell is a mammalian cell.

The presently-disclosed subject matter includes of producing an antibody or an antigen-binding portion thereof that specifically binds pathologic forms of calcineurin. In some embodiments, the method includes (a) culturing a host cell as disclosed herein under conditions that allow said antibody or antigen-binding portion to be expressed, wherein the host cell comprises nucleotide sequences coding the heavy chain and light chain of the antibody or antigen-binding portion, and (b) isolating said antibody or antigen-binding portion from the culture.

The presently-disclosed subject matter includes or antigen fragment thereof. In some embodiments, the antibody is selected from the group consisting of: (a) an antibody comprising the sequence of SEQ ID NO: 3; (b) an antibody comprising the sequence of SEQ ID NO: 5, (c) an antibody comprising the sequence of SEQ ID NOs: 3 and 5; (d) an antibody comprising the sequence of SEQ ID NO: 7; (e) an antibody comprising the sequence of SEQ ID NO: 9; (f) an antibody comprising the sequence of SEQ ID NOs: 7 and 9.

The presently-disclosed subject matter includes a hybridoma cell line deposited with American Type Culture Collection (ATCC) under deposit number PTA-126750 or PTA-126751. The presently-disclosed subject matter includes an antibody expressed by such a hybridoma cell line. The presently-disclosed subject matter includes a method of producing an antibody or an antigen-binding portion thereof that specifically binds pathologic forms of calcineurin, which involves: (a) culturing such a hybridoma cell line, and (b) isolating said antibody or antigen-binding portion from the culture.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1C include the results of a Western blot screen of mouse antisera against full-length and 4CN.

FIG. 2 includes the results of initial screens of hybridoma supernatants. Each Western blot includes: lane 1, full length CN, lane 2, Δ48 kDa CN, and lane 3 MWS.

FIG. 3 includes the results of a Western blot of mAB final subclones.

FIG. 4 includes the results of a Western blot of new 4CN Monoclonal antibodies. Lane 1: Membrane-cytosol fraction 5×FAD Mice, which has full length CN. Lane 2: Nuclear Fraction 5×FAD Mice, which has Δ48 kDa CN. Lane 3: Molecular Weight Standards.

FIG. 5A includes results showing that the monoclonal antibody 17E1 detects astrocytes in 5×FAD but not wild type mice.

FIG. 5B includes results showing that the monoclonal antibody 26A6 does not stain 5×FAD or wild type brain tissue.

FIG. 6 includes results showing that 26A6 reacts to classic calpain-cleaved calcineurin site, while 17E1 detects a cleavage site unique to 5× tissue.

FIGS. 7A and 7B include results confirming that 17E1 mAb and Rb N-terminal Ab are both detecting 48 kDa calcineurin.

FIG. 8A-8C. Mouse monoclonal antibody 26A6, preferentially binds 48 kDa ΔCN fragment. FIG. 8A, Cartoons of full-length CN (CN) and 48 kDa and 45 kDa calpain-generated CN cleavage fragments are shown, depicting location of catalytic domain, CN B binding domain (CNB), Ca²⁺ activated calmodulin binding domain, and autoinhibitory domain (AID). The site of calpain cleavage was previously determined by mass spectrometry analysis (Wu, Tomizawa et al. 2004). The immunizing peptide is shown, with C-terminal sequence from 48 kDa shown in red. FIG. 8B, Results of 26A6 in ELISA to immunizing peptide, and isotype of monoclonal antibody are shown. FIG. 8C, Pure human CN (Hu CN) or endogenous mouse CN (Ms CN) were cleaved by pure human calpain 1 or 2 (clpn 1/2) in the presence or absence of calmodulin (calmod). The left panel shows digestion with calpain 1, and the right panel shows digestion with calpain 2. The top blot shows the detection of CN with the rabbit N-terminal CN antibody in the red channel (which detects all CN forms), and the detection with the mouse monoclonal 26A6 in the green channel. The overlap of detection by the red and green channels gives rise to a bright yellow band only for the 48 kDa form of CN. A black and white of only the green channel is also shown, to more clearly demonstrate the bands specifically detected by 26A6 (26A6 BW). The 26A6 antibody detects the 48 kD band of the pure human CN to a much higher degree than a faint reactivity to the full-length CN. In addition, the 26A6 antibody detects the endogenous mouse brain CN cleaved with calpain 2, as indicated by the black arrow in the black and white panel.

FIG. 9A-9C. Mouse monoclonal antibody 26A6 shows higher levels of labeling in brain tissue with pathologies. FIG. 9A, Examples of human brain with known pathologies that have been labeled with 26A6 antibody (green), GFAP (red) and DAPI nuclear label (blue) indicated that a large number of astrocytes co-labeled with 26A6 and GFAP in the diseased tissue (yellow in the overlay, indicated by single white arrowheads). The top set of panels showed examples at lower magnification and the bottom panels showed examples with higher magnification. FIG. 9B, An example of a normal human brain section did not label with 26A6 antibody. FIG. 9C, There was heterogeneity of 26A6 labeling of astrocytes—some astrocytes labeled with GFAP and 26A6 (yellow in overlays, single arrowheads), others labeled for 26A6, but not GFAP (indicated by double arrowheads in middle panels), while others were positive for GFAP, but did not label strongly for 26A6 (double arrows, in lower panels). Taken together, these data show that 26A6 robustly labels a subset of astrocytes in tissue with pathologies.

FIG. 10. Mouse monoclonal antibody 26A6 labeling is displaced by the immunizing peptide as a competitor. Using a case that had especially robust staining with 26A6 antibody, a competition assay was carried out to provide evidence that the 26A6 antibody was selectively labeling Δ48 kDa CN in the human brain sections. 26A6 antibody was divided into 2 matching aliquots, and immunizing peptide (75 ug/ml) was added to one half. These were incubated at room temperature for 30 minutes, and then these aliquots were used for overnight staining of human brain sections, in the same manner as for FIG. 9A-9C. The competitor displaced almost all the 26A6 signal (with peptide, green 26A6 panel).

FIG. 11A-11B. There is robust expression of Δ48 CN around amyloid plaques and throughout AD tissue. FIG. 11A, A particularly high pathology AD case was selected for analysis with amyloid β 1-42 antibody (red channel), 26A6 (green channel) and GFAP (blue channel). There were numerous amyloid-labeled plaques and a blood vessel in cross section. Surrounding these features, there were a number of GFAP and 26A6 co-labeled cells, best seen as teal cells in the overlay and indicated by white arrowheads. There were a few green cells that did not label with GFAP, but the majority of the 26A6-labeled cells were GFAP-positive astrocytes. FIG. 11B, To extend the analysis of this tissue, sensitive colorimetric IHC was also carried out using DAB-labeling of 26A6 containing cells in brown and SG-labeling of β-amyloid in blue. Again, there was robust peppering of Δ48 CN containing cells around the plaques and blood vessels (BV), but there is also expression of Δ48 CN in areas devoid of plaques and blood vessels in the near vicinity. Quantification in FIG. 12.

FIG. 12. Quantitative assessment of Δ48 CN around amyloid plaques and throughout AD tissue from multiple cases. Multiple Alzheimer's Disease (AD) cases (see Table 2, cases R5409, R5200, R5332, R5329, R1259) and non-demented controls (ND, R5434, R5356, 5500, 5501 and 5450) were co-stained for 26A6 and amyloid β plaques, as shown in FIG. 11B for case R5200. The average from 10 fields of view (FOV) from each case was were randomly acquired. Areas with and without Abeta plaques were captured from AD samples. For image analysis, the entire set of images were converted to 8 bit, then background subtraction, and thresholding were homogeneously performed using FIJI software. Number of positive staining, area, and sum intensity were measured and statistically processed using GraphPad Prism. Although there was some variability in AD cases, the level of 26A6 staining was considerably higher than ND controls. The difference between regions with and without plaques was not significant, indicating that Δ48 CN is expressed throughout AD tissue.

FIG. 13. 6 Δ48 CN Ab labels infarcts. The SMTG region of case 5444, a case with cerebrovascular disease (CVD) that was known to have large infarcts, was used to analyze the reactivity of the 26A6 antibody to this type of feature. The 26A6 antibody labels a large number of cells intensely within an infarct, and many of these co-stain with GFAP, best seen in the overlay (yellow, examples indicated by arrowheads), although there are also 26A6-positive cells that do not stain with GFAP (examples indicated by double arrowheads). In regions away from the infarct, there are not nearly as many 26A6 labeled cells, although the ones that are present also co-label with GFAP.

### BRIEF DESCRIPTION OF THE SEQUENCE LISTING

SEQ ID NO: 1 includes the polypeptide sequence of the C-terminal end of a 48 kDa fragment of the proteolyzed calcineurin as disclosed herein.

SEQ ID NO: 2 includes the nucleic acid sequence encoding the polypeptide sequence of the heavy chain of an antibody that is referred to herein as 17E1.

SEQ ID NO: 3 includes the polypeptide sequence of the heavy chain of an antibody that is referred to herein as 17E1.

SEQ ID NO: 4 includes the nucleic acid sequence encoding the polypeptide sequence of the light chain of an antibody that is referred to herein as 17E1.

SEQ ID NO: 5 includes the polypeptide sequence of the light chain of an antibody that is referred to herein as 17E1.

SEQ ID NO: 6 includes the nucleic acid sequence encoding the polypeptide sequence of the heavy chain of an antibody that is referred to herein as 26A6.

SEQ ID NO: 7 includes the polypeptide sequence of the heavy chain of an antibody that is referred to herein as 26A6.

SEQ ID NO: 8 includes the nucleic acid sequence encoding the polypeptide sequence of the light chain of an antibody that is referred to herein as 26A6.

SEQ ID NO: 9 includes the polypeptide sequence of the light chain of an antibody that is referred to herein as 26A6.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Disclosed herein are unique and advantageous antibodies or antigen-binding portions thereof that specifically binds pathologic forms of calcineurin. The presently-disclosed subject matter includes antibodies, or antigen-binding portions thereof, which specifically bind to pathologic forms of calcineurin. The invention further provides a method of obtaining such antibodies and nucleic acids encoding the same. The invention further relates to compositions and methods for use of these antibodies.

The practice of the presently-disclosed subject matter will employ, unless otherwise indicated, conventional techniques of molecular biology (including recombinant techniques), microbiology, cell biology, biochemistry and immunology, which are within the skill of the art. Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The presently-disclosed subject matter includes monoclonal antibodies that bind pathologic delta-CN fragments, but do not bind full-length (normal) CN. In conjunction with immunohistochemistry, antibodies of the instant invention can pinpoint where delta-CN is generated in relation to different cell types and/or developing neuropathology.

The presently-disclosed subject matter provides antibodies, or antigen-binding portions thereof, which specifically bind to pathologic forms of calcineurin. The invention further provides a method of obtaining such antibodies and nucleic acids encoding the same. The invention further relates to compositions and methods for use of these antibodies.

The presently-disclosed subject matter includes isolated nucleic acid molecules. In some embodiments, the isolated nucleic acid molecule encoding a heavy chain, a light chain, or both a heavy chain and a light chain of an antibody or an antigen-binding portion thereof that specifically binds pathologic forms of calcineurin. In some embodiments, the isolated nucleic acid molecule comprises: (a) the nucleotide sequence of SEQ ID NO: 2; (b) the nucleotide sequence of SEQ ID NO: 4; (c) the nucleotide sequence pairs SEQ ID NOs: 2 and 4; (d) the nucleotide sequence of SEQ ID NO: 6; (e) the nucleotide sequence of SEQ ID NO: 8; or (f) the nucleotide sequence pairs SEQ ID NOs: 6 and 8.

The presently-disclosed subject matter includes vectors. In some embodiments, the vector comprises a nucleic, acid as disclosed herein. The presently-disclosed subject matter includes host cells. In some embodiments, the host cell comprises a nucleic acid as disclosed herein or a vector as disclosed herein. In some embodiments, the host cell is a mammalian cell.

The presently-disclosed subject matter includes of producing an antibody or an antigen-binding portion thereof that specifically binds pathologic forms of calcineurin. In some embodiments, the method includes (a) culturing a host cell as disclosed herein under conditions that allow said antibody or antigen-binding portion to be expressed, wherein the host cell comprises nucleotide sequences coding the heavy chain and light chain of the antibody or antigen-binding portion, and (b) isolating said antibody or antigen-binding portion from the culture.

The presently-disclosed subject matter includes or antigen fragment thereof. In some embodiments, the antibody is selected from the group consisting of: (a) an antibody comprising the sequence of SEQ ID NO: 3; (b) an antibody comprising the sequence of SEQ ID NO: 5, (c) an antibody comprising the sequence of SEQ ID NOs: 3 and 5; (d) an antibody comprising the sequence of SEQ ID NO: 7; (e) an antibody comprising the sequence of SEQ ID NO: 9; (f) an antibody comprising the sequence of SEQ ID NOs: 7 and 9.

The presently-disclosed subject matter includes a hybridoma cell line deposited with American Type Culture Collection (ATCC) under deposit number PTA-126750 or PTA-126751. The presently-disclosed subject matter includes an antibody expressed by such a hybridoma cell line. The presently-disclosed subject matter includes a method of producing an antibody or an antigen-binding portion thereof that specifically binds pathologic forms of calcineurin, which involves: (a) culturing such a hybridoma cell line, and (b) isolating said antibody or antigen-binding portion from the culture.

As will be appreciated by one of ordinary skill in the art, and as disclosed herein, an antibody or antigen fragment thereof, as disclosed herein, has a variety of utilities. For example, the antibody or antigen fragment can be used in tissue staining methods.

CN is known to be a Ca²⁺/calmodulin-dependent protein phosphatase that is ubiquitously expressed in most mammalian tissues, but found at especially high levels in brain. It is also known that Ca²⁺ dysregulation is found in brain aging, Alzheimer's disease (AD) and other AD-related dementias is thought to disrupt cellular signaling and increase vulnerability to disease through many pathways, including those regulated by CN. Many studies have shown that expression and/or activity levels of CN are elevated in human brain tissue at early and late stages of AD. Pathologic forms of CN have been observed in human AD tissue and may appear very early in the disease process. The antibodies and antigen fragments disclosed herein specifically binds pathologic forms of CN.

Accordingly, the presently-disclosed subject matter is inclusive of a method for staining pathological tissue. Such method can be used to stain mammalian tissue, including human tissue. Such method can be used to stain brain tissue. Due to specificity to which the antibodies and antigen fragments disclosed herein bind pathologic forms of CN, they have utility for staining AD tissue. Given the observations in the art that pathologic forms of CN may appear very early in the AD disease process, such a method has utility not only in connection with AD diagnosis, but in earlier-stage diagnosis of AD and/or other dementias.

Staining methods that make use of antibodies and antibody fragments are known in the art and can include, for example, the following.

Immunofluorescence is a method that involves the use of fluorophore-conjugated antibodies to visualize specific proteins within tissues or cells. When excited by specific wavelengths of light, the fluorophores emit fluorescence, allowing for the precise localization of the target antigens.

Immunoperoxidase staining (IHC-P) is a method in which antibodies labeled with enzymes, such as horseradish peroxidase (HRP), bind to specific antigens. The enzyme catalyzes a reaction with a chromogenic substrate, resulting in the formation of a colored precipitate at the site of antibody-antigen binding. This color can be visualized under a microscope.

Immunoenzymatic staining (e.g., alkaline phosphatase) is a method similar to immunoperoxidase staining. This method uses enzyme-conjugated antibodies, such as alkaline phosphatase. The enzyme reacts with a chromogenic substrate to produce a visible color, allowing for the identification of the target antigen.

Immunohistology on frozen sections is a method in which tissues are frozen and sectioned before applying fluorophore-conjugated antibodies. This method is useful for preserving the native state of antigens and is often employed when studying certain types of tissues.

Immunogold labeling (electron microscopy) is a technique used in electron microscopy and involves the use of gold nanoparticles conjugated to antibodies. The gold particles provide contrast in electron micrographs, allowing for the ultrastructural localization of specific antigens.

Although not commonly used in connection with staining tissues, Enzyme-Linked Immunosorbent Assay (ELISA) techniques also have great utility and can also make use of the antibodies and antigen fragments as disclosed herein. Accordingly, as will be appreciated by one of ordinary skill in the art upon study of this document, the antibodies and antigen fragments as disclosed herein have a variety of utilities in connection with methods in the research, characterization, and diagnostic settings.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this application.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

An “antibody” is an immunoglobulin molecule capable of specific binding to a target, such as a carbohydrate, polynucleotide, lipid, polypeptide, etc., through at least one antigen recognition site, located in the variable domain of the immunoglobulin molecule. As used herein, the term encompasses not only intact polyclonal or monoclonal antibodies, but also, unless otherwise specified, any antigen-binding portion thereof that competes with the intact antibody for specific binding, fusion proteins comprising an antigen-binding portion, and any other modified configuration of the immunoglobulin molecule that comprises an antigen recognition site. Antigen-binding portions include, for example, Fab, Fab′, F(ab′)2, Fd, Fv, domain antibodies (dAbs, e.g., shark and camelid antibodies), portions including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. Depending on the antibody amino acid sequence of the constant region of its heavy chains, immunoglobulins can be assigned to different classes. There are five major classes (i.e., isotypes) of immunoglobulins: IgA, IgD, IgE, IgG, and IgM, and several of these may be further divided into subclasses (subtypes), e.g., IgG₁, IgG₂, IgG₃, IgG₄, IgA₁ and IgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known.

The terms “antigen-binding portion” or “antigen-binding fragment” of an antibody (or simply “antibody portion”), as used interchangeably herein, refers to one or more portions of an antibody that retain the ability to specifically bind to an antigen (e.g., delta-CN). It has been shown that the antigen-binding function of an antibody can be performed by portions of a full-length antibody.

A “variable domain” of an antibody refers to the variable domain of the antibody light chain (VL) or the variable domain of the antibody heavy chain (VH), either alone or in combination. As known in the art, the variable domains of the heavy and light chains each consist of four framework regions (FRs) connected by three complementarity determining regions (CDRs) also known as hypervariable regions, and contribute to the formation of the antigen-binding site of antibodies. If variants of a subject variable domain are desired, particularly with substitution in amino acid residues outside a CDR (i.e., in the framework region), appropriate amino acid substitution, in some embodiments, conservative amino acid substitution, can be identified by comparing the subject variable domain to the variable domains of other antibodies which contain CDR1 and CDR2 sequences in the same canonical class as the subject variable domain.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

The term “antigen (Ag)” refers to the molecular entity used for immunization of an immunocompetent vertebrate to produce the antibody (Ab) that recognizes the Ag or to screen an expression library (e.g., phage, yeast or ribosome display library, among others). Herein, Ag is termed more broadly and is generally intended to include target molecules that are specifically recognized by the Ab, thus including portions or mimics of the molecule used in an immunization process for raising the Ab or in library screening for selecting the Ab.

Generally, the term “epitope” refers to the area or region of an antigen to which an antibody specifically binds, i.e., an area or region in physical contact with the antibody. Thus, the term “epitope” refers to that portion of a molecule capable of being recognized by and bound by an antibody at one or more of the antibody's antigen-binding regions.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. Also, an antibody “specifically binds” or “preferentially binds” to a target if it binds with greater affinity, avidity, more readily, and/or with greater duration to that target in a sample than it binds to other substances present in the sample. As such, “specific binding” or “preferential binding” does not necessarily require (although it can include) exclusive binding.

A variety of assay formats may be used to select an antibody or peptide that specifically binds a molecule of interest. For example, solid-phase ELISA immunoassay, immunoprecipitation, Biacore™ (GE Healthcare, Piscataway, N.J.), KinExA, fluorescence-activated cell sorting (FACS), Octet™ (ForteBio, Inc., Menlo Park, Calif) and Western blot analysis are among many assays that may be used to identify an antibody that specifically reacts with an antigen or a receptor, or ligand binding portion thereof, that specifically binds with a cognate ligand or binding partner.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected and/or transformed in vivo with a polynucleotide of this disclosure.

As used herein, “vector” means a construct, which is capable of delivering, and, in some embodiments, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. In some embodiments, diluents for aerosol or parenteral administration are phosphate buffered saline (PBS) or normal (0.9%) saline. Compositions comprising such carriers are formulated by well-known conventional methods.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

Monoclonal antibodies to Δ48 kDa Calcineurin were prepared as follows. Immunization was carried out by Genscript using the peptide CGGGESVLTLK (SEQ ID NO: 1), which represents the C-terminal end of a 48 kDa fragment of proteolyzed calcineurin. This peptide was conjugated to KLH and injected into three (3) Balb/c mice and three (3) C57 Mice.

ELISA Screens were carried out by Genscript to the peptide CGGGESVLTLK (SEQ ID NO: 1). Western screens to endogenous proteolyzed Δ48 kDa calcineurin were carried out using extracts from 2 year old 5×FAD mice brains, which have exceptionally high amounts of this fragment.

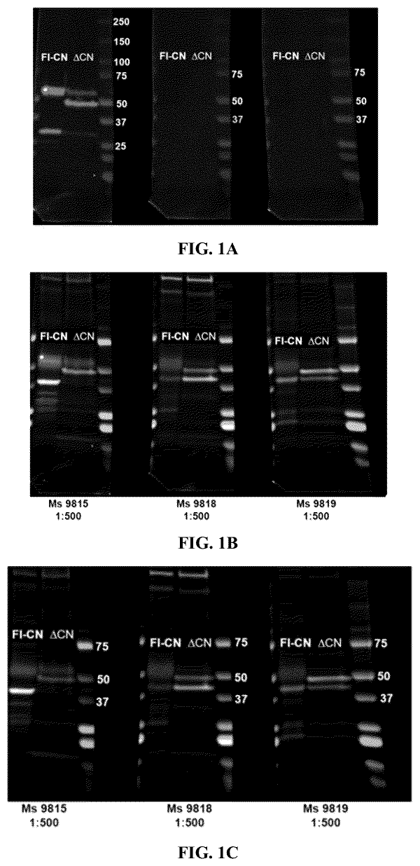

Initial ELISA screens of the mouse antisera gave low results, which is likely due to the inability of the mice to make an antibody to a sequence so similar to their endogenous sequence. However, a Western screen was conducted and turned out well. Two immunoreactivities of interest were identified, one at 48 kDa and one at 45 kDa. Because of these Western results, it was possible to get a Δ48 kDa-specific calcineurin antibody. The Western data are provided in FIGS. 1A-1C, and the ELISA data are provided in Table 1.

TABLE 1
Results of Initial ELISA Screen of Mice

| Animal Number | Signal in 1:1000 dilution |
|---|---|
| #9815 | 0.333 |
| #9816 | 0.086 |
| #9817 | 0.058 |
| #9818 | 1.499 |
| #9819 | 0.612 |
| #9820 | 0.108 |

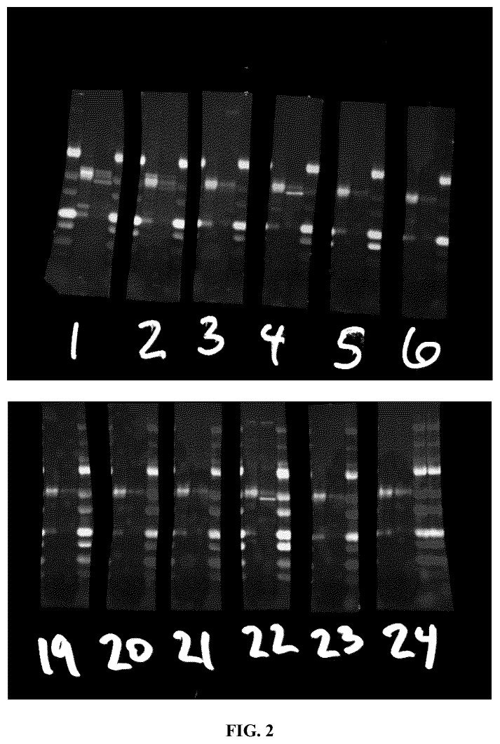

Animals 9818 and 9819 were selected to take forward into hybridoma fusion. Hybridoma supernatants were screened in “mini” western blots. Each western blot included: lane 1, full length CN; lane 2, Δ48 kDa CN; and lane 3 MWS. A total of 48 supernatants were screened, and the two (2) positives selected are shown in FIG. 2. The antibody shown in lane 4 is 17E1 and the antibody shown in lane 22 is 26A6. Western screening was repeated. The final positive subclone screen and selection of the final antibodies was conducted.

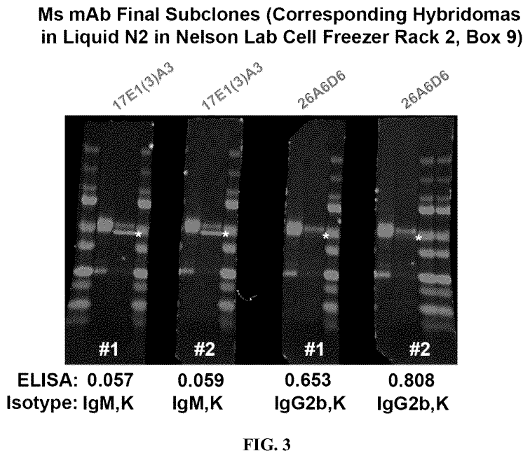

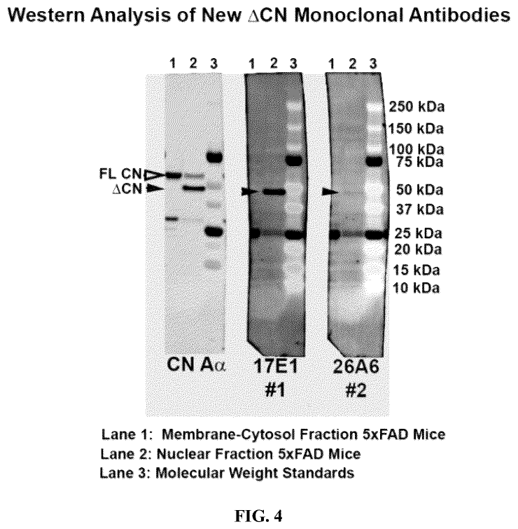

FIG. 3 shows IgG heavy chains in western blots. A problem with these blots is that the IgG heavy chain that runs at 50 kDa undermines the appearance of the blots in terms of showing the appearance of the 60 kDa versus 48 kDa bands. Therefore, future blots were conducted with a light chain-specific second antibody. With reference to FIG. 4, subsequent blots more clearly show that the monoclonal antibodies do not see anything but the 48 kDa band (the 25 kDa band is the antibody light chain). As shown in FIG. 4, it is clear that the clones do not react with the 60 kDa full length calcineurin. Monoclonal antibody 17E1 is very strong in the Western, while 26A6 is stronger in the ELISA.

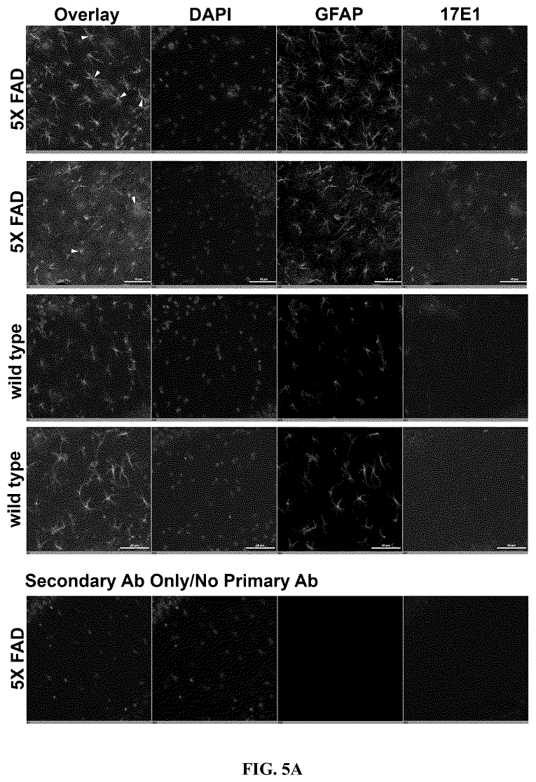

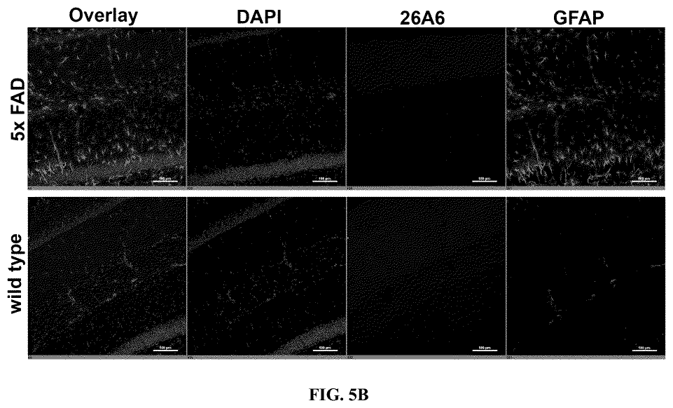

With reference to FIG. 5A, monoclonal antibody 17E1 detects astrocytes in 5×FAD but not wild type mice. 12-14 month old 5×FAD and wild type littermate controls were perfused with ice cold saline and brains harvested into 4% paraformaldehyde. After 24 hrs, the brains were transferred to 30% sucrose until saturated, sectioned in 40 μm sections, and stored in antifreeze at −20 C until used for labeling. Following optimization of staining including antigen retrieval, the following were shown: DAPI in blue, rabbit anti-GFAP (Cell Signaling) in green, and protein L-purified 17E1 mAb (— 10 ug/ml) in red (although FIG. 5A is present in greyscale). The GFAP antibody was counterstained with 488-conjugated goat anti-rabbit and the 17E1 with 594-conjugated goat anti-mouse.

The 17E1 mAb consistently stained 5× brain tissue, but not wild type littermate control brain tissue. 5×FAD brain tissue treated with and secondary antibodies only shows no staining. The 17E1 antibody does not stain all astrocytes uniformly. The white arrows in the overlay indicate astrocytes with yellow centers indicating especially strong co-staining with GFAP and 17E1 antibodies. There are also astrocytes that have relatively low levels of 17E1 expression. Spatial gene profiling techniques can be used to determine how the transcriptional signature in 17E1-stained cells differ from those that do not express it. It is possible that 17E1 marks astrocytes that are distressed.

With reference to FIG. 5B, monoclonal antibody 26A6 does not stain 5×FAD or wild type brain tissue. The same tissue used for the staining carried out in panel A was stained with affinity-purified mAb 26A6. In this case, the 26A6 staining was done in the green channel since the analysis was carried out with a tyramide signal amplification kit (Invitrogen Superboost Kit, 488-conjuated goat anti-mouse). The GFAP was counterstained with 594-conjugated anti-rabbit in the red channel. No signal was detected, even with this signal amplification approach (and multiple tries with various antigen-retrieval approaches). Although signal was not detected with the 26A6 antibody under these conditions and in this type of tissue, it could be used in other types of neurodegenerative conditions.

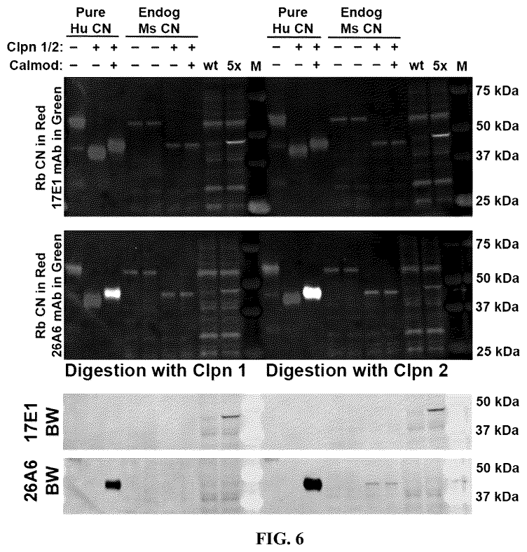

With reference to FIG. 6, it was determined that 26A6 reacts to classic calpain-cleaved calcineurin site, while 17E1 detects a cleavage site unique to 5× tissue. The peptide which was used as an antigen was based on the cleavage site of the 48 kDa calpain-cleavage product of calcineurin reported by Wu et al., Critical Role of Calpain-mediated Cleavage of Calcineurin in Excitotoxic Neurodegeneration, JBC 279: 4929-4940, 2004. This site was also cleaved in vitro by calpain, in the presence of calmodulin (Wu et al., 2004). To confirm that these mAbs reacted with the cleavage site to which they were targeted, both recombinant human calcineurin (CN) and endogenous mouse brain calcineurin were taken, and cleavage was performed with purified calpain 1 or calpain 2 (Clpn 1 or 2). For pure human calcineurin, the inclusion of purified calmodulin altered the cleavage site, as reported previously (Wu et al., 2004), while the endogenous mouse brain calcineurin was 48 kDa in both the presence and absence of added calmodulin. This result may indicate that endogenous calmodulin was associated with the endogenous mouse calcineurin. 26A6 only detected the 48 kDa form of calcineurin, consistent with the cleavage site reported previously (ending at K424) and consistent with its observed reactivity to the peptide ESVLTLK424 with which the animal was immunized. Surprisingly, mAb 17E1 did not react with the calcineurin cleaved in vitro with calpain 1 or calpain 2, but did bind the 48 kDa band present in5×FAD (5×) tissue. Looking closely at the gel, it is apparent that the band identified by 17E1 is slightly larger than that bound by 26A6. It is contemplated that the 17E1-detected fragment is post-translationally altered, e.g., the C-terminus may be acetylated on the lysine or phosphorylated on either the serine or threonine.

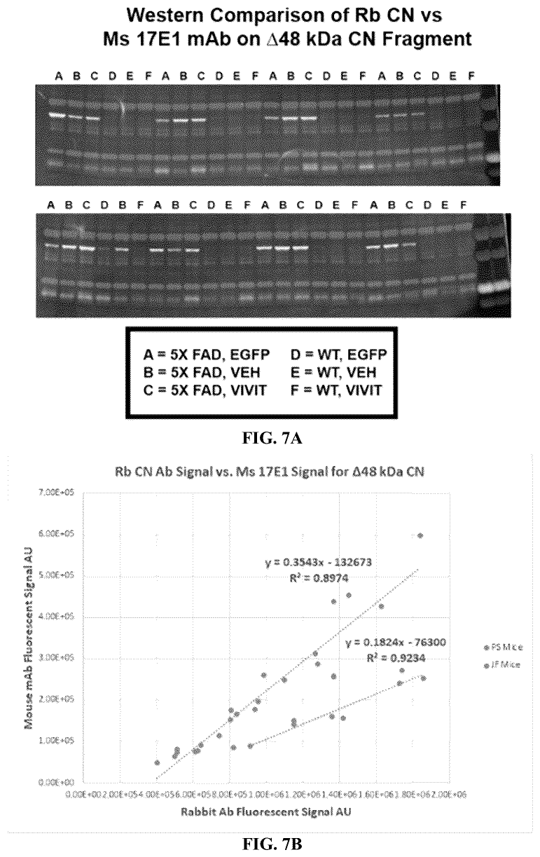

With reference to FIG. 7, co-linear detection of 17E1 mAb Target and Rb N-terminal Ab was conducted. To provide support that the 48 kDa band detected by the 17E1 mAb and the Rb calcineurin N-terminal antibody are the same protein, panels of 5×FAD versus wild type tissue were probed with both antibodies. Two sets of mice were used for this experiment. There was co-linear expression of the fragments, as shown by the high R² values, indicating that these antibodies appear to be detecting the same protein fragment.

### Example 2

Calcineurin (CN) is a Ca²⁺/calmodulin-dependent protein phosphatase. In healthy tissue, CN exists mainly as a full-length (˜60 kDa) highly-regulated protein involved in essential cellular functions. However, in diseased or injured tissue, CN is proteolytically converted to a constitutively active fragment that has been causatively-linked to numerous pathophysiologic processes. These calpain-cleaved CN fragments (ΔCN) appear at high levels in human brain at early stages of cognitive decline associated with Alzheimer's disease (AD).

As described herein, a monoclonal antibody to ΔCN was developed using an immunizing peptide corresponding to the C-terminal end of the ΔCN fragment.

A mouse monoclonal antibody was obtained, designated 26A6, that selectively detects ΔCN in Western analysis of calpain-cleaved recombinant human CN. Using this antibody, pathological and normal human brain sections provided by the University of Kentucky's Alzheimer's Disease Research Center were screened. 26A6 showed low reactivity towards normal brain tissue, but detected astrocytes both surrounding AD amyloid plaques and throughout AD brain tissue. In brain tissue with infarcts, there was considerable concentration of 26A6-positive astrocytes within/around infarcts, suggesting a link with anoxic/ischemia pathways.

The monoclonal 26A6 antibody is highly selective for the ΔCN proteolytic fragment and labels a subset of astrocytes, and provides a useful tool for marking insidious brain pathology and identifying novel astrocyte phenotypes.

Immunization and Development of Hybridoma Lines.

3 Balb/c and 3 C57Bl/6 mice were immunized with the peptide antigen CGGGESVLTLK (SEQ ID NO: 1), based on the Lys 424 calpain-dependent cleavage site (Wu et al., 2004, Pleiss et al., 2016), as a KLH conjugate. After 3 boosts, serum was tested in a Western blot, and initial screens indicated that 2 of the mice had substantial selective reactivity towards the proteolyzed CN band. These mice were selected for further boosting and fusion with a Sp2/0 myeloma partner. The resulting hybridomas were screened first in ELISA assays to the immunizing peptide and secondly in Western blot assays to the uncleaved and cleaved CN, to confirm that they selectively bound the cleaved CN. A positive clone was identified-26A6. This clone was developed/subcloned further and characterized as to isotype. 26A6 is an IgG2b with a kappa light chain.

This hybridoma line was cultured. The culture medium was high glucose DMEM supplemented with 10% heat-inactivated fetal bovine serum, 2 mM glutamine, 1 mM sodium pyruvate, 100 units/ml penicillin, and 300 ug/ml streptomycin. The cultures are maintained in a 6% CO2 cell culture incubator. Culture medium is collected and either used directly at dilutions ranging from 1:2 to 1:5 or the antibodies from the culture supernatants purified on Protein G as described below.

ELISA Assays.

ELISA assays were carried out at GenScript. The immunizing peptide itself was bound to plates at a concentration of 1 μg/ml, 100 μl per well and supernatants from the hybridomas were used to bind to the peptide. An anti-IgG-HRP conjugated secondary antibody was used to detect the primary 26A6 antibody.

Calpain cleavage of CN in vitro and characterization of 26A6 binding to fragments by Western analysis.

To assess the preference of the 26A6 antibody for the cleaved form of CN relative to the full-length form, cleavage of CN with calpain was carried out in vitro and the ability of 26A6 to bind full-length versus cleaved fragments assessed in Western blots. All cleavage reactions were carried out in CN dilution buffer (4 mM CaCl₂), 5 mM DTT and 50 mM Tris, pH 7.5). Purified human CN was from Enzo Life Sciences (Cat. #BML-SE163, Farmingdale, NY). This CN is a recombinant human CN with a 60 kDa Aα chain and a 15 kDa CN B chain in a buffer containing 50 mM Tris, pH 7.5, 100 mM NaCl, 6 mM MgCl₂, 5 mM DTT, 0.025% NP40, and 0.5 mM CaCl₂. Endogenous mouse brain CN was prepared from the cytosolic fraction of wild type mouse brain extracts, desalted on Zeba columns (Cat. #89882, Thermofisher, Waltham, MA) to remove protease inhibitors and exchange into CN dilution buffer. Both purified human and desalted endogenous mouse CN are stable when stored at −80. Purified calmodulin (Cat. #BML-SE325, Enzo Life Sciences, Farmingdale, NY) was dissolved into CN dilution buffer at a concentration of 1 μg/μl, then aliquoted and stored at −80. Purified human calpain 1 (Cat. #C6108, Sigma, St. Louis, MO) was received in a buffer of 5 mM Beta mercaptoethanol, 1 mM EDTA, 1 mM EGTA, 30% Glycerol, 20 mM Imidazole hydrochloride at a concentration of 1 unit/111. Purified recombinant rat calpain 2 was kindly provided by Dr. Dorothy Croall from the University of Maine. It was in a buffer of 50 mM MOPS, pH 7.5, 1 mM EDTA, 1 mM EGTA and 0.5 mM DTT. The cleavage reactions were carried out by combining 5 ul of human or endogenous mouse CN with 5 ul of calmodulin (or buffer control) and 5 ul of 1:10 diluted calpain 1 or 1:5 diluted calpain 2. The calpain 1 reactions were heated at 37 C for 15 minutes. The calpain 2 reactions were heated at 28 C for 15 minutes. After calpain treatment, samples were quenched in 2× gel loading buffer and heated at 65 C for 30 minutes prior to gel and Western blot analysis.

Following denaturation, full-length or proteolyzed protein samples were resolved on 4-20% gradient gels (Criterion gels #5671095, BioRad, Hercules, Ca) and transferred to PVDF membranes. As a positive control in Westerns, a commercially-available rabbit polyclonal antibody to the amino terminus of CN (1:2500, Cat. No. 07-1492, Millipore, Burlington, MA) was used, which detects both the full-length (60 kDa) and proteolyzed fragments (48 kDa, 45 kDa). The rabbit primary is detected by a 680RD anti-rabbit secondary (P/N 926-68071, LI-COR, Lincoln, NE) and the 26A6 mouse monoclonal is detected by a 790 anti-mouse light chain secondary (Cat. No. 115-655-174, JacksonlmmunoResearch, West Grove, PA). It is important to use a secondary directed at the mouse antibody light chain, since the 50 kDa antibody heavy chain in samples can lead to interference with data assessment. Westerns were imaged on an Odyssey Scanner (LI-COR, Lincoln, NE) and the quantification was performed using Image Studio 2.1 Software (LI-COR; RRID:SCR 013715). Data is shown with both the green (800) and red (680) channels together, resulting in a yellow signal where there is overlapping detection, or individual channels are shown in black and white.

Purification of Antibodies.

The 26A6 antibody was purified from antibody supernatants using Protein G agarose (Cat. No. 20398, Pierce BioTech, Waltham, MA). Cell culture supernatants were diluted 1:1 with phosphate buffered saline, pH 7.2, and run over columns containing the resin. The resin was washed with 10 column volumes of phosphate buffered saline, and eluted with a buffer containing 0.1 M glycine, pH 2.8. The eluate was collected into fractions with 1/10 volume of Tris, pH 8.8 buffer. Immediately, A280 readings were taken on a spectrophotometer (DU 730, Beckman Coulter) and the peak fractions pooled. The samples were dialyzed against cold phosphate buffered saline, pH 7.2 overnight at 4° C. The column was regenerated by washing with 10 volumes of the glycine elution buffer and re-equilibrated with phosphate buffered saline containing 0.05% sodium azide for storage at 4° C. The column could be re-used multiple times. The next morning following dialysis, a final protein reading was taken, samples were taken for gel analysis of purity, and the antibodies aliquoted and stored at −80° C.

Human brain sections preparation and staining.

Human brain sections were obtained from the University of Kentucky Alzheimer's Disease Research Center (UK-ADRC) Tissue Repository, with samples provided as numbered specimens. A total of 7 different disease cases, either AD or CVD, were examined and 5 different age-matched controls. All subjects were participants in the UK-ADRC Autopsy program, and the postmortem time interval for all subjects used in these studies is approximately 3 hrs. (Nelson, Jicha et al. 2007, Abdul, Sama et al. 2009, Mohmmad Abdul, Baig et al. 2011). These samples were formaldehyde-fixed, paraffin-embedded sections cut into ˜8 μm thickness and mounted on glass microscope slides. Slides were baked in a 40° C. oven overnight, then deparaffinized using SafeClear (Fisher Scientific, Hampton, NH), and rehydrated through a series of alcohols and finally into PBS. Antigen retrieval was carried out in pressurized decloaking chamber (BioCare Medical, Concord, CA), according to manufacturer's directions using the Borg antigen retrieval solution (BioCare Medical, Concord, CA). Following antigen retrieval, slides were washed 3× in PBS. For sections designated for staining with the β-amyloid antibody downstream, the sections were treated with 70% formic acid for 5 mins, then washed. All sections were treated with 3% H₂O₂ in methanol to block endogenous peroxide activity. Slides were washed in PBS and sections were either stained for immunofluorescence or immunohistochemistry.

For immunofluorescence, sections were incubated in blocking solution (PBS with 3% bovine serum albumin, 5% goat serum, and 0.05% Triton X 100) for 30 minutes, and incubated overnight at room temperature in primary antibody in that same blocking solution. Primary antibodies used were: rabbit anti-GFAP (1:200; catalog #12389, Cell Signaling Technology, Danviers, MA) and mouse 26A6 hybridoma supernatants diluted 1:5 or 10 ug/ml of purified 26A6 antibody. Sometimes 75 μg/ml immunizing peptide was included with the 26A6 antibody as a negative control. Following washes in PBS, primary antibodies were incubated with 594-conjugated goat anti-rabbit (1:200, Invitrogen, Waltham, MA), biotinylated anti mouse (Tyramide Superboost Amplification kit, Invitrogen, Waltham, MA), and DAPI (1:1000, ThermoFisher, Waltham, MA). Slides were washed in PBS and the biotinylated anti-mouse antibody signal was amplified using the 488-conjugated tyramide reagent according to the manufacturer's directions in the Tyramide Superboost Amplification kit. Alternatively, sometimes sections were stained with 26A6 and rabbit antibody to amyloid β 1-42 (1:1000, ab201060, Abcam, Cambridge, UK). The sections were then counterstained the mouse 488 Tyramide Superboost kit and 594-conjugated goat anti-rabbit. Subsequently, sections were stained with rat monoclonal antibody to GFAP (1:200, 13-0300, Invitrogen) and counterstained with 405-conjugated goat anti-rat antibody (1:200, ab175671, Abcam, Cambridge, UK). Slides were washed and mounted in EverBrite True Black Hardset Mounting Medium (23017, Biotium, Fremont, CA). Fluorescent confocal microscope images were taken using a Nikon Eclipse Ti microscope (Minato City, Tokoyo, Japan).

For immunohistochemistry, sections were blocked in PBS with 3% bovine serum albumin, 5% goat serum, 5% horse serum, and 0.05% Triton X 100. Incubation of sections with 26A6 antibody and rabbit antibody to amyloid β 1-42 (1:1000, Abcam, ab201060) was done as described above. Secondary antibodies and avidin-biotin complex (ABC) amplifications used were from VectaStain Elite ABC-HRP kits (mouse PK-6102 and rabbit PK-6101, Vector Laboratories, Newark, CA), according to manufacturer's directions. The mouse 26A6 antibody was detected with the brown DAB substrate and the rabbit amyloid β 1-42 antibody was detected with the blue SG substrate (Vector Labs). Following dehydration in alcohols and SafeClear, the sections were mounted in DPX mounting medium (100504-938, VWR, Radnor, PA) and imaged on a Nikon Eclipse 90i.

Quantification and Statistical Analysis of HRP-labeled sections.

After immunohistochemistry staining, ten field of views (FOV) of gray matter from each human brain sample were randomly acquired using a 60× objective (Nikon Eclipse 90i). Areas with and without Aβ plaques were captured from AD samples. For image analysis, the entire set of images were converted to 8 bit, then background subtraction, and thresholding were homogeneously performed using FIJI software. Number of positive staining, area, and sum intensity were measured and statistically processed using GraphPad Prism.

Monoclonal Antibody, 26A6, Shows Greater Reactivity to 48 kDa CN Fragment (Δ48 CN)

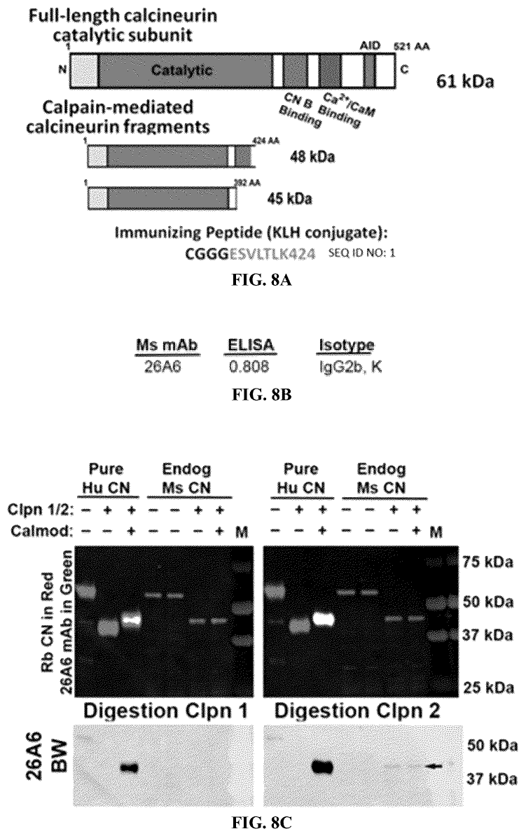

Full-length CN is susceptible to cleavage by the endogenous Ca²⁺-activated protease, calpain, at several sites (Wang, Villalobo et al. 1989, Wu, Tomizawa et al. 2004) and FIG. 8A). Some of the resulting fragments, a 48 kDa fragment and a 45 kDa fragment, lack an autoinhibitory domain (AID), causing these fragments to become constitutively active (see FIG. 1A). Previous work indicated that the 48 kDa CN fragment (Δ48 CN) in particular, was associated with postmortem human AD brain samples (Mohmmad Abdul, Baig et al. 2011, Pleiss, Sompol et al. 2016), and therefore the same peptide antigen CGGGESVLTLK (SEQ ID NO: 1) was used to immunize mice with, as was used previously to generate a polyclonal rabbit antibody (Pleiss, Sompol et al. 2016). This immunizing peptide corresponds to the C-terminus of Δ48 CN (FIG. 8A).

A number of hybridomas that were positive were obtained in the initial ELISA screen to the peptide antigen (total of −64 wells), including monoclonal antibody 26A6 (FIG. 8B). However, secondary screens in Western blots to proteolyzed CN revealed only one antibody that selectively detected Δ48 CN in Western analysis as well (FIG. 8C). Mouse monoclonal antibody, 26A6, bound the 48 kDa calpain-cleaved CN with a high degree of specificity, showing very little reactivity towards either the full-length CN or the 445 CN (FIG. 8C). There did seem to be some preference for CN cleaved by calpain 2 over calpain 1, as the endogenous mouse brain CN Δ48 CN was detected when cleaved by the former and not the latter, as was seen most clearly in the 26A6 black and white exposure (FIG. 8C). Having developed an antibody reagent that selectively detects the Δ48 CN in Westerns, the issues of what cells expressed this fragment in vivo and with what pathologies these cells were associated could be addressed using immunostaining approaches.

The 26A6 Monoclonal Detects Δ48 CN to a Higher Degree in Astrocytes of High Pathology Human Brain Tissue

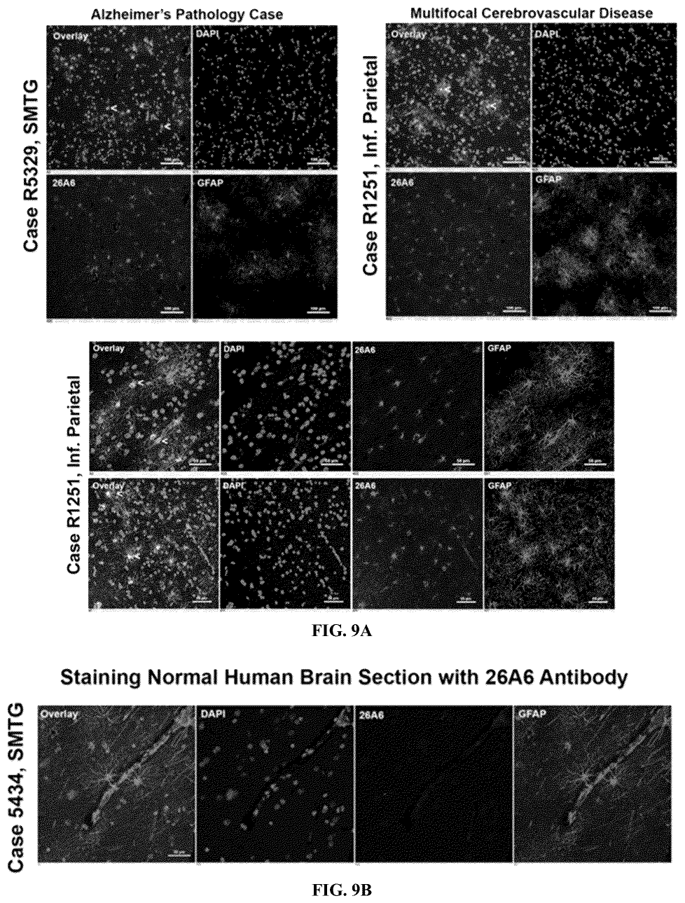

The initial labeling experiments utilized human brain sections from patients with known pathologies (see Table 2). Patients had dementia with underlying Alzheimer's disease (AD) and/or cerebrovascular disease (CVD), or were clinically normal. Initially, samples were co-stained with the 26A6 monoclonal and an antibody to glial fibrillary acid protein (GFAP), a well-known marker of astrocytes. As shown for cases R5329 and R1251 in FIG. 9A, there was considerable co-labeling of cells with these two markers, seen best as the yellow overlap between the two fluorescent labels in the overlay. These data indicated that many of the 26A6 labeled, and therefore Δ48 CN-containing, cells were astrocytes. The low magnification images showed a robust scattering of 26A6-labeled cells in both pathological samples (FIG. 9A), while there was no discernable labeling with 26A6 in the normal brain specimen, case 5434 (FIG. 9B). The higher magnification images (FIG. 9A, lower panel and FIG. 9C) showed that the 26A6 label was centrally located in the cell body rather than penetrating out into the astrocytic processes the way that GFAP labeling does. In addition, there was considerable heterogeneity in the 26A6 labeling. This was best shown in FIG. 9C. Only a subset of astrocytes labeled with 26A6, and there were a few cells that stained with 26A6 and not GFAP. Primarily, however, it seemed 26A6 labeled a subset of astrocytes in brain tissue with pathologies.

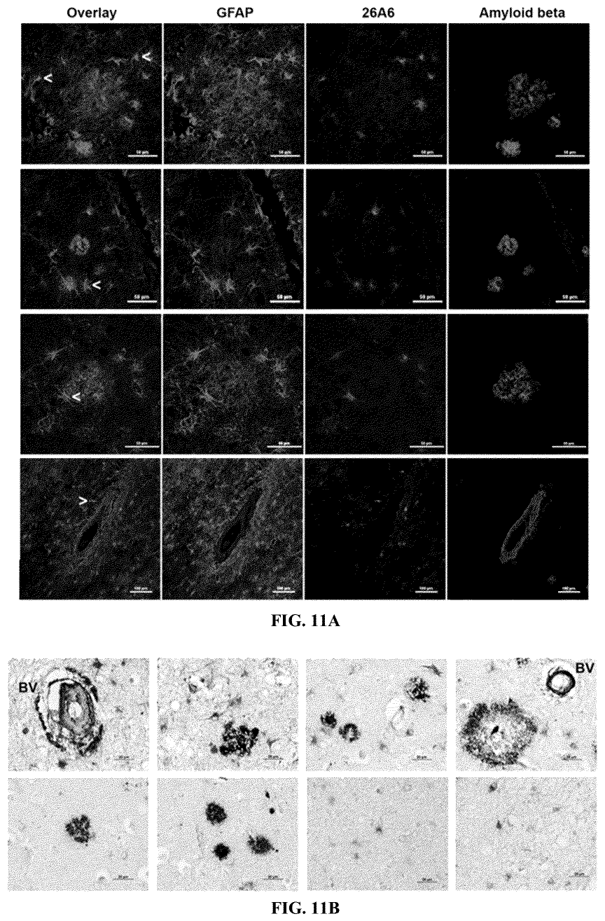

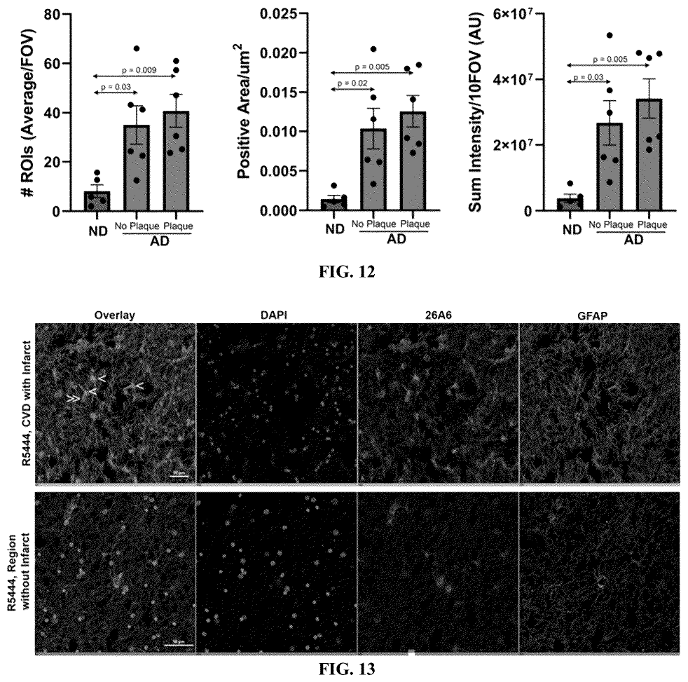

Table 2 includes a summary of the cases used in this report. Cases 5329, 1251, and 5434 are shown in FIG. 9A-9C. Case R5200 is shown in FIGS. 3 and 4. Cases R5434, R5356, 5500, 5501 and 5450 (Normal) and R1259, R5329, R5332, R1251, R5200, and R5409 (Demented) were analyzed and quantified in FIG. 12. Case R5444, containing a large infarct, is shown in FIG. 13.

TABLE 2
Human Brain Tissue Used for Screening Mouse Monoclonal Antibody 26A6

| | Age | | | | | | |
|---|---|---|---|---|---|---|---|
| Case | at | | | Last Clinical | | | |
| No. | Death | Sex | APOE | Index | Notes | ADCERAD | Consensus Dx |
| R5444 | 86 | M | e3/e3 | MCI | 1) AD-low level | A = | CVD + ARTAG |
| | | | | | 2) CVD, moderate | CERAD | |
| | | | | | with large infarct | possible | |
| | | | | | 3) ARTAG | | |
| | | | | | 4) Mild Vermian | | |
| | | | | | Atrophy, Cerebellum | | |
| R5434 | 75 | M | e3/e4 | NORMAL | 1) ARTAG, | No | ARTAG |
| | | | | | widespread | | |
| | | | | | 2) CVD-mild | | |
| | | | | | 3) NO AD | | |
| | | | | | Pathology | | |
| R5500 | 89 | F | e3/e3 | NORMAL | 1) Normal | No | Not done yet |
| | | | | | 2) Low AD | | |
| | | | | | Pathology | | |
| | | | | | 3) LB pathology, | | |
| | | | | | Olf. Bulb only | | |
| | | | | | 4) CVD-Mild | | |
| R5501 | 83 | M | e3/e4 | NORMAL | 1) Normal | No | Not done yet |
| | | | | | 2) Low AD | | |
| | | | | | Pathology | | |
| | | | | | 3) CVD-Mild | | |
| R5450 | 83 | F | e3/e3 | NORMAL | 1) PART-Definite | No | Normal + LATE |
| | | | | | 2) CVD-mild | | |
| | | | | | 3) LATE-Stage 2 | | |
| R5356 | 94 | M | e2/e3 | NORMAL | Low AD Pathology | NO | Normal |
| R5409 | 99 | F | e3/e3 | DEMENTED | AD-High Level | YES | AD + HS + |
| | | | | | | | ARTAG + |
| | | | | | | | CVD |
| R5200 | 91 | F | e3/e3 | DEMENTED | AD | YES | AD + CVD + |
| | | | | | | | HS |
| R1251 | 82 | M | e3/e4 | DEMENTED | Multifocal CVD | C = | AD + CVD |
| | | | | | | Definition | |
| | | | | | | AD | |
| R5332 | 83 | M | e3/e3 | DEMENTED | AD | C = | AD + CVD |
| | | | | | | Definition | |
| | | | | | | AD | |
| R5329 | 86 | F | e3/e4 | DEMENTED | High AD Pathology | C = | AD + DLB + |
| | | | | | | Definition | CVD |
| | | | | | | AD | |
| R1259 | 87 | F | e3/e4 | DEMENTED | AD | C = | AD + CVD |
| | | | | | | Definition | |
| | | | | | | AD | |

APOE (apolipoprotein E) gene, ADCERAD (Semiquantitative measure of neuritic plaques), MCI (mild cognitive impairment), AD (Alzheimer's Disease), CVD (cerebrovascular disease), ARTAG (aging-related tau astrogliopathy), LB (Lewy body), PART (primary age-related tauopathy), LATE (limbic predominant age-related TDP-43 encephalopathy).

The Immunizing Peptide Displaces 26A6 Labeling, Indicating Specificity of 26A6 Binding to Δ 48CN

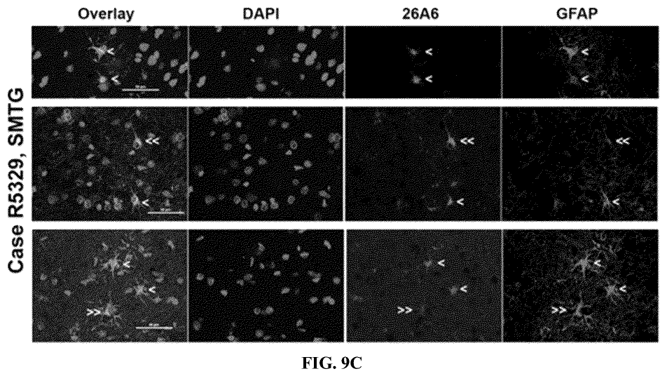

In order to confirm that the labeling detected by the 26A6 monoclonal antibody in immunostaining was truly due to the presence of Δ 48CN in the tissue, a competition analysis was carried out using the immunizing peptide to displace 26A6 labeling in the tissue of a particularly high pathology case (FIG. 10). As was clearly shown, the cellular label by 26A6 was displaced in the presence of the peptide, while there was strong labeling in the control tissue.

Δ48 CN is Found Both Around Plaques and Distributed Throughout Brain in AD Tissue

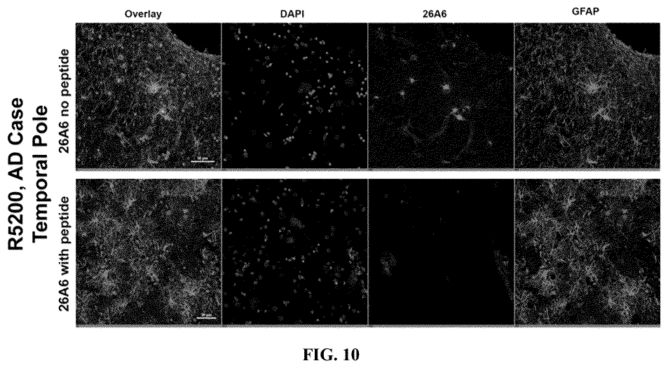

A particularly high pathology AD case, R5200, was examined in detail by immunofluorescent and colorimetric immunohistochemical staining techniques. As shown in FIG. 11A, brain sections were probed with GFAP, 26A6 and an antibody to amyloid β 1-42. The amyloid antibody detected clear plaques, and in the bottom row of FIG. 4A, a blood vessel in cross-section. Around these amyloid-stained features, 26A6 and GFAP co-labeled astrocytes, best observed as a teal color in the overlay. It was noteworthy that only a subset of astrocytes contain the A48 CN, as there were more cells that express GFAP alone than both 26A6 and GFAP. Nevertheless, a high level of primarily astrocytes expressing A48 CN were present in AD tissue. To look at this tissue more broadly, this same case was stained for both 26A6 and amyloid β 1-42 using sensitive colorimetric immunohistochemical techniques in FIG. 11B. Again there was a robust peppering of labeled 26A6 cells in brown, as well as both amyloid-labeled plaques and blood vessels in blue. It is noteworthy that there was prominent 26A6-staining around plaques, but not all plaques are surrounded by 26A6 labeled cells. In addition, there were plaque-free regions of tissue that still have robust 26A6 labeling (e.g. see FIG. 11B). In FIG. 12, a quantitative assessment A48 CN distribution from several AD cases was carried out. Although there was case-to-case variablity, on the whole there was far greater concentration of Δ48 CN in AD cases compared to neglible amounts in non-demented controls. In general, there was robust expression of Δ48 CN both around plaques and throughout the AD tissue.

Δ48 CN is Enriched in Regions of Infarcts within Brain Tissue from Subjects with Cerebrovascular Disease

Since previous work with the polyclonal antibody to ΔCN had shown especially robust labeling of cerebral infarcts (Pleiss, Sompol et al. 2016), a case from a subject with known infarcts was examined to determine how the monoclonal would react to this same type of feature (FIG. 13). Within the expanse of the infarct, there was considerable 26A6 staining, and many of these 26A6 positive cells co-labeled with GFAP (seen as yellow in overlay). In regions outside the infarct, there were fewer 26A6-positive cells, although these also co-labeled with GFAP. Taken together, these data indicated that there is an association of 4CN with and around infarcts.

The 26A6 Antibody is a Unique Tool for Assessing the Presence of Proteolyzed CN in Disease

Most of the commercially available antibodies to CN are targeted against the N-terminus or the C-terminus of the protein. Both have their advantages and disadvantages. Antibodies directed towards the N-terminus are able to detect both the full-length CN and all forms of CN cleaved by calpain (Wang, Villalobo et al. 1989, Wu, Tomizawa et al. 2004, Wu, Tomizawa et al. 2007), including the constitutively active 48 kDa and 45 kDa forms. While these N-terminal antibodies can distinguish between CN forms in Western analysis because of the molecular weight differences on the gels, they cannot distinguish between them in immunostaining, and therefore the utility of these antibodies for this purpose is limited. Antibodies directed to the C-terminus of CN (e.g., Abcam ab71149, Sigma C1956 or Cell Signaling 2614) are even more limited since they cannot detect proteolyzed CN even in Westerns, and thus are only useful for detecting full-length CN. The use of such antibodies may not reveal contributions of the proteolyzed form during brain disease processes (Billingsley, Ellis et al. 1994, Sidoli, Reed et al. 2021). A rabbit polyclonal antibody to the Δ48 CN was previously produced and the data obtained were quite similar to that provided herein (Pleiss, Sompol et al. 2016), but like all polyclonal antibodies, there was a limited supply. To overcome this problem, the same peptide-base immunization strategy that was previously used to make the polyclonal was also used to create a monoclonal—the 26A6 monoclonal.

The most attractive feature of the 26A6 monoclonal is that it, like the previous rabbit polyclonal, selectively detects the Δ48 CN (Pleiss, Sompol et al. 2016) and FIG. 8A-8C). Proteolysis of CN occurs both under in vitro conditions and also in intact nervous tissue after cellular insult (Wu, Tomizawa et al. 2004, Huang, Fileta et al. 2005, Shioda, Moriguchi et al. 2006, Shioda, Han et al. 2007, Mohmmad Abdul, Baig et al. 2011, Rosenkranz, May et al. 2012). Although this proteolysis has been widely documented, only the advent of the antibodies directed to the 48 kDa C-terminus has allowed us to probe where these proteolysis products accumulate. Comparison of the immunological staining results obtained with the rabbit polyclonal and this new 26A6 monoclonal are strikingly similar. Both antibodies preferentially stain brain tissue from subjects with AD or CVD/infarcts; both antibodies stain robustly around amyloid plaques but also throughout AD tissue; both antibodies preferentially stain in and around infarcts. Although both antibodies stain a small number of non-astrocytic cells, they primarily stain a subset of astrocytes. It's not clear why 26A6 apparently exhibits low levels of neuronal labeling, as neurons can also show extensive Ca²⁺ dysregulation with AD, and CN is known to become hyperactive in experimental models of AD (e.g. Dineley, Hogan et al 2007, Hudry, Wu et a12012, Hopp, Bihlmeyer et al 2018). Moreover, calpains have been shown to cleave CN in primary neurons (Wu, Tomizawa et al 2004). Several possibilities for differential labeling of astrocytes and neurons in the present study include: (1) 26A6 selectively detects the 48 kDa species of CN and may have missed other calpain-mediated fragments (e.g. 57 and 45 kDa) that may occur at similar or higher levels in neurons. (2) Calpains may have greater access to CN in astrocytes in vivo, or CN proteolysis may be associated with pathological processes that are unique to astrocytes. (3) Ca²⁺ dysregulation and CN proteolysis may emerge at different disease stages in astrocytes and neurons. For instance, assessment of human cases at the MCI stage of AD may have revealed similar or higher levels of 4 CN48 labeling in neurons. Finally, significant CN proteolysis may be happening in neurons, but it is restricted to dendritic spines (and other micro compartments), where CN and calpains are known to interact, and, as such, may be below the detection methods used here.

Δ48 CN is Likely an Endogenous Marker of Distressed Astrocytes

A broad body of work indicates that the Δ48 CN is a marker of distressed astrocytes. Both the Δ48 CN and the Δ45 CN lack the autoinhibitory domain (AID) of the CN A chain, and therefore are constitutively active phosphatases that are no longer regulated by calcium (Wang, Villalobo et al. 1989, Wu, Tomizawa et al. 2004, Wu, Tomizawa et al. 2007, Kraner and Norris 2018, Sompol and Norris 2018). It is noteworthy that the Δ48 CN is only produced in the presence of calmodulin, whether added to the pure human CN samples or present in the endogenous mouse brain extracts (FIG. 8A-8C). In the absence of calmodulin, the calpain-cleavage generates the Δ45 CN (FIG. 8A-8C). These observations are consistent with those observed previously—that cleavage into the Δ48 CN requires calmodulin (Wang, Villalobo et al. 1989, Wu, Tomizawa et al. 2004). Work from others has shown that the Δ48 CN is found in brain extracts from AD tissue (Mohmmad Abdul, Baig et al. 2011), and the 26A6 monoclonal is very selective for this form.

High levels of CN proteolysis can dramatically alter cell function and viability (Wang, Pathan et al. 1999, Wu, Tomizawa et al. 2004, Mohmmad Abdul, Baig et al. 2011). In cultured astrocytes, expression of the 4CN leads to the production and release of a variety of cytokines and chemokines linked to neuroinflammation and glial activation (Sama, Mathis et al. 2008). Astrocytes infected with 4CN overexpressing viruses also take on a reactive, hypertrophied morphology (Norris, Kadish et al. 2005). Similar to effects in neurons (Wu, Hudry et al. 2010, Hopp, Bihlmeyer et al. 2018), expression of 4CN in astrocytes of hippocampus has been shown to reduce synaptic strength (Pleiss, Sompol et al. 2016), suggesting that 4CN in astrocytes leads to impaired neurologic function. However, others have suggested that astrocytic 4CN could have some beneficial effects in brain, as well (Fernandez, Jimenez et al. 2012). Clearly, additional work is required to assess the nuances of 4CN signaling in astrocytes associated with neurodenenerative conditions, and the 26A6 antibody could be a useful tool for these investigations.

Distressed Astrocytes and Different Brain Pathologies

The pattern of Δ48 CN-expressing astrocytes was somewhat different in different brain pathologies. In AD, amyloid-positive plaques and blood vessels were robustly surrounded by these astrocytes, but there was also a peppering of labeled astrocytes throughout brain tissue in these cases (FIG. 11A-11B). In addition, there was variability in the degree to which different AD-positive cases expressed Δ48 CN-positive astrocytes (FIG. 12). Tissue from control subjects had negligible labeling (FIG. 8B and FIG. 12). These data are similar to those observed previously with the rabbit polyclonal antibody (Pleiss, Sompol et al. 2016) and may account for the elevated levels of CN and CN-dependent signaling mediators, such as NFATs, reported in previous data sets for both mouse models of AD and related dementias, as well as human disease cases (Wu, Tomizawa et al. 2004, Reese, Zhang et al. 2008, Abdul, Sama et al. 2009, Mohmmad Abdul, Baig et al. 2011, Serrano-Perez, Martin et al. 2011, Neria, del Carmen Serrano-Perez et al. 2013, Caraveo, Auluck et al. 2014).

Previous studies have shown that CN proteolytic fragments appear with both stroke/global ischemia (Shioda, Moriguchi et al. 2007, Rosenkranz, May et al. 2012) as well as microinfarcts (Pleiss, Sompol et al. 2016). Consistent with these observations, the present study observed extensive co-labeling of high cerebral pathology cases with 26A6, especially in GFAP-positive cells within infarcted regions (FIG. 13). However, labeling was highly variable and numerous GFAP-negative/Δ48 CN positive cells were also prominent. The high level of Δ48 CN expression with infarcts may contribute to high levels of morbidity and co-morbidity in dementia and suggests a link between Δ48 CN and anoxic/ischemic processes (Kraner and Norris 2018, Price, Norris et al. 2018). These data were somewhat similar to those found previously with a rabbit polyclonal antibody to Δ48 CN (Pleiss, Sompol et al. 2016, Kraner and Norris 2018), except the polyclonal antibody appeared to label areas within the infarct as well as the apparent scar-forming astrocytes on the periphery of the infarct. These difference could be due to recognition of both 45 and 48 kDa species of 4CN by the polyclonal antibody. Interestingly, the polyclonal antibody also tended to label more neurons in diseased tissue than 26A6.

Increasingly, it is recognized that infarcts and microinfarcts contribute to vascular dementia, either by themselves or in conjunction with other neurodegenerative diseases such as AD (O'Brien, Erkinjuntti et al. 2003, Kalaria, Akinyemi et al. 2012, van Norden, van Dijk et al. 2012, Raz, Daugherty et al. 2015, Nelson, Trojanowski et al. 2016, Vemuri and Knopman 2016, Wilcock, Schmitt et al. 2016, Corriveau, Koroshetz et al. 2017) (Price, Norris et al. 2018, Shih, Hyacinth et al. 2018). It has been shown that hyperactive CN signaling arising from amyloid pathology, cerebrovascular pathology, and other sources exacerbates neurodegenerative processes and hastens cognitive decline (Reese and Taglialatela 2011, Furman and Norris 2014, Kraner and Norris 2018, Sompol and Norris 2018, Price, Johnson et al. 2021). Moreover, mounting evidence gathered from preclinical models suggests that the inhibition of CN signaling, either through genetic or pharmacologic approaches, ameliorates numerous pathophysiologic and cognitive phenotypes of AD and ADRDs (e.g. Dineley, Hogan et al. 2007, Dineley, Kayed et al. 2010, Wu, Hudry et al. 2010, Hudry, Wu et al. 2012, Furman, Sama et al. 2012, Sompol, Furman et al. 2017, Radhakrishnan, Ubele et al. 2021, Sompol, Gollihue et al. 2021, Sompol, Gollihue et al. 2023). This work is consistent with recent epidemiologic studies showing that use of FDA approved CN inhibitors is associated with a reduced prevalence of dementia in human populations (Taglialatela, Rastellini et al. 2015, Silva, Taglialatela et al. 2023). Thus, tools for identifying the appearance of proteolytic forms of CN (e.g. the monoclonal antibody 26A6) could be very important for determining which pathologies may benefit from CN-inhibiting strategies.

Conclusions. The new monoclonal 26A6 is highly selective for the Δ48 kDa CN proteolytic fragment and labels a subset of astrocytes and, possibly other cell types, under pathological conditions. In brain tissue with infarcts, there was considerable concentration of 26A6-positive astrocytes within/around infarcts, suggesting a link with anoxic/ischemic pathways. Cell specific labeling of Δ48 CN with 26A6 opens up the possibility of using newer spatial profiling technologies to discover how these cells differ from those which do not express this protein, and perhaps confirm the effects of cell Ca²⁺ changes on a cell-by-cell basis.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

Abdul, H. M., M. A. Sama, J. L. Furman, D. M. Mathis, T. L. Beckett, A. M. Weidner, E. S. Patel, I. Baig, M. P. Murphy, H. LeVine, 3rd, S. D. Kraner and C. M. Norris (2009). “Cognitive decline in Alzheimer's disease is associated with selective changes in calcineurin/NFAT signaling.” J Neurosci 29(41): 12957-12969.
Aramburu, J., A. Rao and C. B. Klee (2000). “Calcineurin: from structure to function.” Curr Top Cell Regul 36: 237-295.
Baumgartel, K. and I. M. Mansuy (2012). “Neural functions of calcineurin in synaptic plasticity and memory.” Learn Mem 19(9): 375-384.
Billingsley, M. L., C. Ellis, R. L. Kincaid, J. Martin, M. L. Schmidt, V. M. Lee and J. Q. Trojanowski (1994). “Calcineurin immunoreactivity in Alzheimer's disease.” Exp Neurol 126(2): 178-184.
Canellada, A., B. G. Ramirez, T. Minami, J. M. Redondo and E. Cano (2008). “Calcium/calcineurin signaling in primary cortical astrocyte cultures: Rcan1-4 and cyclooxygenase-2 as NFAT target genes.” Glia 56(7): 709-722.
Caraveo, G., P. K. Auluck, L. Whitesell, C. Y. Chung, V. Baru, E. V. Mosharov, X. Yan, M. Ben-Johny, M. Soste, P. Picotti, H. Kim, K. A. Caldwell, G. A. Caldwell, D. Sulzer, D. T. Yue and S. Lindquist (2014). “Calcineurin determines toxic versus beneficial responses to alpha-synuclein.” Proc Natl Acad Sci USA 111(34): E3544-3552.
Corriveau, R. A., W. J. Koroshetz, J. T. Gladman, S. Jeon, D. Babcock, D. A. Bennett, S. T. Carmichael, S. L. Dickinson, D. W. Dickson, M. Emr, H. Fillit, S. M. Greenberg, M. L. Hutton, D. S. Knopman, J. J. Manly, K. S. Marder, C. S. Moy, C. H. Phelps, P. A. Scott, W. W. Seeley, B. A. Sieber, N. B. Silverberg, M. L. Sutherland, A. Taylor, C. L. Torborg, S. P. Waddy, A. K. Gubitz and D. M. Holtzman (2017). “Alzheimer's Disease-Related Dementias Summit 2016: National research priorities.” Neurology 89(23): 2381-2391.
Dineley, K. T., D. Hogan, W. R. Zhang and G. Taglialatela (2007). “Acute inhibition of calcineurin restores associative learning and memory in Tg2576 APP transgenic mice.” Neurobiol Learn Mem 88(2): 217-224.
Dineley, K. T., R. Kayed, V. Neugebauer, Y. Fu, W. Zhang, L. C. Reese and G. Taglialatela (2010). “Amyloid-beta oligomers impair fear conditioned memory in a calcineurin-dependent fashion in mice.” J Neurosci Res 88(13): 2923-2932.
Disterhoft, J. F., J. R. Moyer, Jr. and L. T. Thompson (1994). “The calcium rationale in aging and Alzheimer's disease. Evidence from an animal model of normal aging.” Ann N Y Acad Sci 747: 382-406.
Fernandez, A. M., S. Fernandez, P. Carrero, M. Garcia-Garcia and I. Torres-Aleman (2007). “Calcineurin in reactive astrocytes plays a key role in the interplay between proinflammatory and anti-inflammatory signals.” J Neurosci 27(33): 8745-8756.
Fernandez, A. M., S. Jimenez, M. Mecha, D. Davila, C. Guaza, J. Vitorica and I. Torres-Aleman (2012). “Regulation of the phosphatase calcineurin by insulin-like growth factor I unveils a key role of astrocytes in Alzheimer's pathology.” Mol Psychiatry 17(7): 705-718.
Foster, T. C. and C. M. Norris (1997). “Age-associated changes in Ca(2+)-dependent processes: relation to hippocampal synaptic plasticity.” Hippocampus 7(6): 602-612.
Furman, J. L. and C. M. Norris (2014). “Calcineurin and glial signaling: neuroinflammation and beyond.” J Neuroinflammation 11: 158.
Furman, J. L., D. M. Sama, J. C. Gant, T. L. Beckett, M. P. Murphy, A. D. Bachstetter, L. J. Van Eldik and C. M. Norris (2012). “Targeting astrocytes ameliorates neurologic changes in a mouse model of Alzheimer's disease.” J Neurosci 32(46): 16129-16140.
Gibson, G. E. and C. Peterson (1987). “Calcium and the aging nervous system.” Neurobiol Aging 8(4): 329-343.
Hopp, S. C., N. A. Bihlmeyer, J. P. Corradi, C. Vanderburg, A. M. Cacace, S. Das, T. W. Clark, R. A. Betensky, B. T. Hyman and E. Hudry (2018). “Neuronal calcineurin transcriptional targets parallel changes observed in Alzheimer disease brain.” J Neurochem 147(1): 24-39.
Hudry E, Wu H Y, Arbel-Ornath M, Hashimoto T, Matsouaka R, Fan Z, Spires-Jones T L, Betensky R A, Bacskai B J, Hyman B T (2012). “Inhibition of the NFAT pathway alleviates amyloid β neurotoxicity in a mouse model of Alzheimer's disease. J Neurosci 32(9):3176-3192.
Huang, W., J. B. Fileta, A. Dobberfuhl, T. Filippopolous, Y. Guo, G. Kwon and C. L. Grosskreutz (2005). “Calcineurin cleavage is triggered by elevated intraocular pressure, and calcineurin inhibition blocks retinal ganglion cell death in experimental glaucoma.” Proc Natl Acad Sci USA 102(34): 12242-12247.
Kalaria, R. N., R. Akinyemi and M. Ihara (2012). “Does vascular pathology contribute to Alzheimer changes?” J Neurol Sci 322(1-2): 141-147.
Khachaturian, Z. S. (1989). “The role of calcium regulation in brain aging: reexamination of a hypothesis.” Aging (Milano) 1(1): 17-34.
Kraner, S. D. and C. M. Norris (2018). “Astrocyte Activation and the Calcineurin/NFAT Pathway in Cerebrovascular Disease.” Front Aging Neurosci 10: 287.
Landfield, P. W. (1987). “′Increased calcium-current'hypothesis of brain aging.” Neurobiol Aging 8(4): 346-347.
Lim, D., L. Tapella, G. Dematteis, M. Talmon and A. A. Genazzani (2023). “Calcineurin Signalling in Astrocytes: From Pathology to Physiology and Control of Neuronal Functions.” Neurochem Res 48(4): 1077-1090.
Lisman, J. (1989). “A mechanism for the Hebb and the anti-Hebb processes underlying learning and memory.” Proc Natl Acad Sci USA 86(23): 9574-9578.
Liu F, Grundke-Iqbal I, Iqbal K, Oda Y, Tomizawa K, Gong C X (2005). “Truncation and activation of calcineurin A by calpain I in Alzheimer disease brain.” J Biol Chem 280(45):37755-37762.
Malinow, R., D. V. Madison and R. W. Tsien (1988). “Persistent protein kinase activity underlying long-term potentiation.” Nature 335(6193): 820-824.
Mansuy I M, Winder D G, Moallem™, Osman M, Mayford M, Hawkins R D, Kandel E R (1998) “Inducible and reversible gene expression with the rtTA system for the study of memory.” Neuron 21(2):257-265.
Mohmmad Abdul, H., I. Baig, H. Levine, 3rd, R. P. Guttmann and C. M. Norris (2011). “Proteolysis of calcineurin is increased in human hippocampus during mild cognitive impairment and is stimulated by oligomeric Abeta in primary cell culture.” Aging Cell 10(1): 103-113.
Mukherjee, A. and C. Soto (2011). “Role of calcineurin in neurodegeneration produced by misfolded proteins and endoplasmic reticulum stress.” Curr Opin Cell Biol 23(2): 223-230.
Mulkey, R. M., C. E. Herron and R. C. Malenka (1993). “An essential role for protein phosphatases in hippocampal long-term depression.” Science 261(5124): 1051-1055.
Nagamoto-Combs, K. and C. K. Combs (2010). “Microglial phenotype is regulated by activity of the transcription factor, NFAT (nuclear factor of activated T cells).” J Neurosci 30(28): 9641-9646.
Nelson, P. T., G. A. Jicha, F. A. Schmitt, H. Liu, D. G. Davis, M. S. Mendiondo, E. L. Abner and W. R. Markesbery (2007). “Clinicopathologic correlations in a large Alzheimer disease center autopsy cohort: neuritic plaques and neurofibrillary tangles “do count” when staging disease severity.” J Neuropathol Exp Neurol 66(12): 1136-1146.
Nelson, P. T., J. Q. Trojanowski, E. L. Abner, 0. M. Al-Janabi, G. A. Jicha, F. A. Schmitt, C. D. Smith, D. W. Fardo, W. X. Wang, R. J. Kryscio, J. H. Neltner, W. A. Kukull, M. D. Cykowski, L. J. Van Eldik and E. T. Ighodaro (2016). ““New Old Pathologies”: AD, PART, and Cerebral Age-Related TDP-43 With Sclerosis (CARTS).” J Neuropathol Exp Neurol 75(6): 482-498.
Neria, F., M. del Carmen Serrano-Perez, P. Velasco, K. Urso, P. Tranque and E. Cano (2013). “NFATc3 promotes Ca(2+)-dependent MMP3 expression in astroglial cells.” Glia 61(7): 1052-1066.
Nguyen, T. and S. Di Giovanni (2008). “NFAT signaling in neural development and axon growth.” Int J Dev Neurosci 26(2): 141-145.
Norris, C. M. (2014) “Calpain Interactions with the Protein Phosphatase Calcineurin in Neurodegeneration.” In: Dhalla, N., Chakraborti, S. (eds) Role of Proteases in Cellular Dysfunction. Advances in Biochemistry in Health and Disease, vol 8. Springer, New York, NY.
Norris, C. M. (2023). “Is It Time to Repurpose Calcineurin Inhibitors for the Treatment of Cognitive Impairment and Dementia?” J Alzheimers Dis.
Norris, C. M., E. M. Blalock, K. C. Chen, N. M. Porter, O. Thibault, S. D. Kraner and P. W. Landfield (2010). “Hippocampal ‘zipper’ slice studies reveal a necessary role for calcineurin in the increased activity of L-type Ca(2+) channels with aging.” Neurobiol Aging 31(2): 328-338.
Norris C M, Blalock E M, Thibault O, Brewer L D, Clodfelter G V, Porter, NM, Landfield P W (2006) “Electrophysiological mechanisms of delayed excitotoxicity: Positive feedback loop between NMDA receptor current and depolarization-mediated glutamate release.” J Neurophysiol 96(5): 2488-2500.
Norris, C. M., I. Kadish, E. M. Blalock, K. C. Chen, V. Thibault, N. M. Porter, P. W. Landfield and S. D. Kraner (2005). “Calcineurin triggers reactive/inflammatory processes in astrocytes and is upregulated in aging and Alzheimer's models.” J Neurosci 25(18): 4649-4658.
O'Brien, J. T., T. Erkinjuntti, B. Reisberg, G. Roman, T. Sawada, L. Pantoni, J. V. Bowler, C. Ballard, C. DeCarli, P. B. Gorelick, K. Rockwood, A. Burns, S. Gauthier and S. T. DeKosky (2003). “Vascular cognitive impairment.” Lancet Neurol 2(2): 89-98.
Perez-Ortiz, J. M., M. C. Serrano-Perez, M. D. Pastor, E. D. Martin, S. Calvo, M. Rincon and P. Tranque (2008). “Mechanical lesion activates newly identified NFATc1 in primary astrocytes: implication of ATP and purinergic receptors.” Eur J Neurosci 27(9): 2453-2465.
Pleiss, M. M., P. Sompol, S. D. Kraner, H. M. Abdul, J. L. Furman, R. P. Guttmann, D. M. Wilcock, P. T. Nelson and C. M. Norris (2016). “Calcineurin proteolysis in astrocytes: Implications for impaired synaptic function.” Biochim Biophys Acta 1862(9): 1521-1532.
Price, B. R., L. A. Johnson and C. M. Norris (2021). “Reactive astrocytes: The nexus of pathological and clinical hallmarks of Alzheimer's disease.” Ageing Res Rev 68: 101335.
Price, B. R., C. M. Norris, P. Sompol and D. M. Wilcock (2018). “An emerging role of astrocytes in vascular contributions to cognitive impairment and dementia.” J Neurochem 144(5): 644-650.
Radhakrishnan H, Ubele M F, Krumholz S M, Boaz K, Mefford J L, Jones E D, Meacham B, Smiley J, Puskás LG, Powell D K, Norris C M, Stark C E L, Head E (2021) “Tacrolimus Protects against Age-Associated Microstructural Changes in the Beagle Brain.” J Neurosci 41(23):5124-5133.
Raz, N., A. M. Daugherty, A. R. Bender, C. L. Dahle and S. Land (2015). “Volume of the hippocampal subfields in healthy adults: differential associations with age and a proinflammatory genetic variant.” Brain Struct Funct 220(5): 2663-2674.
Reese, L. C. and G. Taglialatela (2011). “A role for calcineurin in Alzheimer's disease.” Curr Neuropharmacol 9(4): 685-692.
Reese, L. C., W. Zhang, K. T. Dineley, R. Kayed and G. Taglialatela (2008). “Selective induction of calcineurin activity and signaling by oligomeric amyloid beta.” Aging Cell 7(6): 824-835.
Rosenkranz, K., C. May, C. Meier and K. Marcus (2012). “Proteomic analysis of alterations induced by perinatal hypoxic-ischemic brain injury.” J Proteome Res 11(12): 5794-5803.
Sama, M. A., D. M. Mathis, J. L. Furman, H. M. Abdul, I. A. Artiushin, S. D. Kraner and C. M. Norris (2008). “Interleukin-lbeta-dependent signaling between astrocytes and neurons depends critically on astrocytic calcineurin/NFAT activity.” J Biol Chem 283(32): 21953-21964.
Serrano-Perez, M. C., E. D. Martin, C. F. Vaquero, I. Azcoitia, S. Calvo, E. Cano and P. Tranque (2011). “Response of transcription factor NFATc3 to excitotoxic and traumatic brain insults: identification of a subpopulation of reactive astrocytes.” Glia 59(1): 94-107.
Schultz B, Taday J, Menezes L, Cigerce A, Leite M C, Gonsalves C A (2021) “Calpain-Mediated Alterations in Astrocytes Before and During Amyloid Chaos in Alzheimer's Disease.” J Alzheimers Dis 84(4):1415-1430.
Shih, A. Y., H. I. Hyacinth, D. A. Hartmann and S. J. van Veluw (2018). “Rodent Models of Cerebral Microinfarct and Microhemorrhage.” Stroke 49(3): 803-810.
Shioda, N., F. Han, S. Moriguchi and K. Fukunaga (2007). “Constitutively active calcineurin mediates delayed neuronal death through Fas-ligand expression via activation of NFAT and FKHR transcriptional activities in mouse brain ischemia.” J Neurochem 102(5): 1506-1517.
Shioda, N., S. Moriguchi, Y. Shirasaki and K. Fukunaga (2006). “Generation of constitutively active calcineurin by calpain contributes to delayed neuronal death following mouse brain ischemia.” J Neurochem 98(1): 310-320.
Shiratori, M., H. Tozaki-Saitoh, M. Yoshitake, M. Tsuda and K. Inoue (2010). “P2X7 receptor activation induces CXCL2 production in microglia through NFAT and PKC/MAPK pathways.” J Neurochem 114(3): 810-819.
Sidoli, M., C. B. Reed, C. Scapin, P. Paez, D. R. Cavener, R. J. Kaufman, M. D'Antonio, M. L. Feltri and L. Wrabetz (2021). “Calcineurin Activity Is Increased in Charcot-Marie-Tooth 1B Demyelinating Neuropathy.” J Neurosci 41(20): 4536-4548.
Silva J D, Taglialatela G, Jupiter D C (2023) “Reduced Prevalence of Dementia in Patients Prescribed Tacrolimus, Sirolimus, or Cyclosporine.” J Alzheimers Dis. doi: 10.3233/JAD-230526. Epub ahead of print.
Sompol, P., J. L. Furman, M. M. Pleiss, S. D. Kraner, I. A. Artiushin, S. R. Batten, J. E. Quintero, L. A. Simmerman, T. L. Beckett, M. A. Lovell, M. P. Murphy, G. A. Gerhardt and C. M. Norris (2017). “Calcineurin/NFAT Signaling in Activated Astrocytes Drives Network Hyperexcitability in Abeta-Bearing Mice.” J Neurosci 37(25): 6132-6148.
Sompol, P., J. L. Gollihue, S. D. Kraner, I. A. Artiushin, R. A. Cloyd, E. A. Chishti, S. A. Koren, G. K. Nation, J. F. Abisambra, O. Huzian, L. I. Nagy, M. Santha, L. Hackler, Jr., L. G. Puskas and C. M.
Norris (2021). “Q134R: Small chemical compound with NFAT inhibitory properties improves behavioral performance and synapse function in mouse models of amyloid pathology.” Aging Cell 20(7): e13416.
Sompol, P., J. L. Gollihue, B. E. Weiss, R. L. Lin, S. L. Case, S. D. Kraner, E. M. Weekman, J. C. Gant, C. B. Rogers, D. M. Niedowicz, T. L. Sudduth, D. K. Powell, A. L. Lin, P. T. Nelson, O. Thibault,
D. M. Wilcock and C. M. Norris (2023). “Targeting Astrocyte Signaling Alleviates Cerebrovascular and Synaptic Function Deficits in a Diet-Based Mouse Model of Small Cerebral Vessel Disease.” J Neurosci 43(10): 1797-1813.
Sompol, P. and C. M. Norris (2018). “Ca(2+), Astrocyte Activation and Calcineurin/NFAT Signaling in Age-Related Neurodegenerative Diseases.” Front Aging Neurosci 10: 199.
Taglialatela, G., C. Rastellini and L. Cicalese (2015). “Reduced Incidence of Dementia in Solid Organ Transplant Patients Treated with Calcineurin Inhibitors.” J Alzheimers Dis 47(2): 329-333.
Thibault, O., J. C. Gant and P. W. Landfield (2007). “Expansion of the calcium hypothesis of brain aging and Alzheimer's disease: minding the store.” Aging Cell 6(3): 307-317.
Uchino, H., Y. Kuroda, S. Morota, G. Hirabayashi, N. Ishii, F. Shibasaki, Y. Ikeda, M. J. Hansson and E. Elmer (2008). “Probing the molecular mechanisms of neuronal degeneration: importance of mitochondrial dysfunction and calcineurin activation.” J Anesth 22(3): 253-262.
van Norden, A. G., E. J. van Dijk, K. F. de Laat, P. Scheltens, M. G. Olderikkert and F. E. de Leeuw (2012). “Dementia: Alzheimer pathology and vascular factors: from mutually exclusive to interaction.” Biochim Biophys Acta 1822(3): 340-349.
Vemuri, P. and D. S. Knopman (2016). “The role of cerebrovascular disease when there is concomitant Alzheimer disease.” Biochim Biophys Acta 1862(5): 952-956.
Vihma, H., P. Pruunsild and T. Timmusk (2008). “Alternative splicing and expression of human and mouse NFAT genes.” Genomics 92(5): 279-291.
Wang, H. G., N. Pathan, I. M. Ethell, S. Krajewski, Y. Yamaguchi, F. Shibasaki, F. McKeon, T. Bobo, T. F. Franke and J. C. Reed (1999). “Ca2+-induced apoptosis through calcineurin dephosphorylation of BAD.” Science 284(5412): 339-343.
Wang, J. H. and P. T. Kelly (1996). “The balance between postsynaptic Ca(2+)-dependent protein kinase and phosphatase activities controlling synaptic strength.” Learn Mem 3(2-3): 170-181.
Wang, K. K., A. Villalobo and B. D. Roufogalis (1989). “Calmodulin-binding proteins as calpain substrates.” Biochem J 262(3): 693-706.
Winder D G, Mansuy I M, Osman M, Moallem™, Kandel E R (1998) “Genetic and pharmacological evidence for a novel, intermediate phase of long-term potentiation suppressed by calcineurin.” Cell 92(1):25-37.
Wilcock, D. M., F. A. Schmitt and E. Head (2016). “Cerebrovascular contributions to aging and Alzheimer's disease in Down syndrome.” Biochim Biophys Acta 1862(5): 909-914.
Wu, H. Y., E. Hudry, T. Hashimoto, K. Kuchibhotla, A. Rozkalne, Z. Fan, T. Spires-Jones, H. Xie, M. Arbel-Ornath, C. L. Grosskreutz, B. J. Bacskai and B. T. Hyman (2010). “Amyloid beta induces the morphological neurodegenerative triad of spine loss, dendritic simplification, and neuritic dystrophies through calcineurin activation.” J Neurosci 30(7): 2636-2649.
Wu, H. Y., K. Tomizawa and H. Matsui (2007). “Calpain-calcineurin signaling in the pathogenesis of calcium-dependent disorder.” Acta Med Okayama 61(3): 123-137.
Wu, H. Y., K. Tomizawa, Y. Oda, F. Y. Wei, Y. F. Lu, M. Matsushita, S. T. Li, A. Moriwaki and H. Matsui (2004). “Critical role of calpain-mediated cleavage of calcineurin in excitotoxic neurodegeneration.” J Biol Chem 279(6): 4929-4940.

### DEPOSIT INFORMATION

Hybridoma cell lines for 17E1 and 26A6 were deposited with the American Type Culture Collection (ATCC), 10801 University Boulevard, Manassas, VA 20110, USA, on Apr. 3, 2020, and given Patent Deposit Nos. PTA-126750 and PTA-126751, respectively. Applicant has or intends to comply with all the requirements set forth in 37 C.F.R. §§ 1.801-1.809.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for staining pathological tissue, comprising:
(a) incubating tissue with an isolated antibody or antigen fragment thereof, wherein the antibody is selected from the group consisting of:
(i) an antibody comprising the sequence of SEQ ID NOs: 3 and 5, or an antibody comprising the sequence of SEQ ID NOs: 7 and 9, or
(ii) an antibody expressed by a hybridoma cell line deposited with American Type Culture Collection (ATCC) under deposit number PTA-126750 or PTA-126751; and
(b) detecting bound antibody, wherein presence of bound antibody indicates presence of pathological tissue.

2. The method of claim 1, wherein the tissue is mammalian tissue.

3. The tissue of claim 2, wherein the tissue is brain tissue.

4. The method of claim 2, wherein the tissue is human tissue.

5. The method of claim 4, wherein the tissue is brain tissue.

6. The method of claim 1, wherein the antibody comprising the sequence of SEQ ID NOs: 3 and 5.

7. The method of claim 1, wherein the antibody comprising the sequence of SEQ ID NOs: 7 and 9.

8. The method of claim 1, wherein the antibody is expressed by the hybridoma cell line deposited with ATCC under deposit number PTA-126750.

9. The method of claim 1, wherein the antibody is expressed by the hybridoma cell line deposited with ATCC under deposit number PTA-126751.


SEQUENCE LISTING (9 sequences)

SEQ ID NO: 1 - protein, 11 aa, synthetic construct
  REGION 1..11: note = Synthesized
CGGGESVLTLK

SEQ ID NO: 2 - DNA, 1779 bp, synthetic construct
  misc_feature 1..1779: note = 17E1 Heavy chain
  source 1..1779: mol_type = other DNA
atgggatggagctggatctttctctttctcctgtcaggaactgcaggtgtcctctctgaggtccagctgcaacaatctggacttgagctggtgaagcctgggggttcagtgaagatatcctgtaaggcttctggatacacgttcactgactattacatgaactgggtgaagcagagccatggaaagagccttgagtggagtggagaaattaatcctaacaatggtgatactaaatacaaccagaaatttaagggcaaggccacattgactgtagacaagtcctccagtacagcctacatggagctccgcagcctgacatctgaggactctgcagtctattactgtgcaagaggggactatggtaaaggatttgcttactggggccaagggactctggtcactgtctctgcagagagtcagtccttcccaaatgtcttccccctcgtctcctgcgagagccccctgtctgataagaatctggtggccatgggctgcctggcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacaaagatctgcatgtgcccattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctattga

SEQ ID NO: 3 - protein, 592 aa, synthetic construct
  REGION 1..592: note = 17E1 Heavy chain
MGWSWIFLFLLSGTAGVLSEVQLQQSGLELVKPGGSVKISCKASGYTFTDYYMNWVKQSHGKSLEWSGEINPNNGDTKYNQKFKGKATLTVDKSSSTAYMELRSLTSEDSAVYYCARGDYGKGFAYWGQGTLVTVSAESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNKDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 4 - DNA, 717 bp, synthetic construct
  misc_feature 1..717: note = 17E1 Light chain
  source 1..717: mol_type = other DNA
atggagaaagacacactcctgctatgggtcctgcttctctgggttccagattccacaggtgacattgtactgacccaatctccagcttctttggctgtgtctctagggcagagggccaccatctcctgcagagccagcgaaagtgttgataattttggctttagttttatgaactggttccaacagaaaccaggacagccacccaaactcctcatctatgctgcatccaagctaggatccggggtccctgccaggtttagtggcagtgggtctgggacagacttcagcctcaacatccatcctatggaggaggatgatactgcaatgtatttctgtcagcaaaataaggaggttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 5 - protein, 238 aa, synthetic construct
  REGION 1..238: note = 17E1 - Light chain
MEKDTLLLWVLLLWVPDSTGDIVLTQSPASLAVSLGQRATISCRASESVDNFGFSFMNWFQQKPGQPPKLLIYAASKLGSGVPARFSGSGSGTDFSLNIHPMEEDDTAMYFCQQNKEVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - DNA, 1422 bp, synthetic construct
  misc_feature 1..1422: note = 26A6 - Heavy chain
  source 1..1422: mol_type = other DNA
atgggatggagctggatctttctcttcctcctgtcaggaactgcaggtgtccactctgaggtccagctgcaacagtctggacctgtgctggtgaagcctggggcttcagtgaagatgtcctgtaaggcttctggatacacattcactgacttctatttgaactgggtgaagcagagccatggaaagacccttgagtggattggagttattaatccttacaacggtattactagatataaccagaagttcgggggcacggccacattgactgttgacaagtcctccagcacagcctacatggaactcaacagcctgacatctgaggactctgcagtctattactgttcaagggggggtaagacctactactttgactactggggccaaggcaccactctcacagtctccccagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 7 - protein, 473 aa, synthetic construct
  REGION 1..473: note = 26A6 - Heavy chain
MGWSWIFLFLLSGTAGVHSEVQLQQSGPVLVKPGASVKMSCKASGYTFTDFYLNWVKQSHGKTLEWIGVINPYNGITRYNQKFGGTATLTVDKSSSTAYMELNSLTSEDSAVYYCSRGGKTYYFDYWGQGTTLTVSPAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 8 - DNA, 720 bp, synthetic construct
  misc_feature 1..720: note = 26A6 - Light chain
  source 1..720: mol_type = other DNA
atgaagttgcctgttaggctgttggtgctgatgttctggattcctgcttccagcagtgatgttgtgatgacccaaagtccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcaacagagtggaggctgaggatctgggagtttatttctgctctcaaagtacacatgttcctccggtcacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 9 - protein, 239 aa, synthetic construct
  REGION 1..239: note = 26A6 - Light chain
MKLPVRLLVLMFWIPASSSDVVMTQSPLSLPVSLGDQASISCRSSQSLVHSNGNTYLHWYLQKPGQSPKLLIYKVSNRFSGVPDRFSGSGSGTDFTLKINRVEAEDLGVYFCSQSTHVPPVTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC